(19) United States
(12) Patent Application Publication
(10) Pub. No.: US 2024/0157363 A1
(43) Pub. Date: May 16, 2024

(54) DIGITAL MICROFLUIDICS-LIKE MANIPULATION OF ELECTROKINETICALLY PRECONCENTRATED BIOPARTICLE/BIOMOLECULE PLUGS IN CONTINUOUS-FLOW

(71) Applicant: Ramot at Tel-Aviv University Ltd., Tel-Aviv (IL)

(72) Inventors: Gilad YOSSIFON, Tel Aviv (IL); Sinwook PARK, Tel Aviv (IL); Barak SABBAGH, Tel Aviv (IL)

(73) Assignee: Ramot at Tel-Aviv University Ltd., Tel-Aviv (IL)

(21) Appl. No.: 18/419,732

(22) Filed: Jan. 23, 2024

Related U.S. Application Data
(63) Continuation of application No. PCT/IL2022/050797, filed on Jul. 24, 2022.
(60) Provisional application No. 63/252,667, filed on Oct. 6, 2021.
(60) Provisional application No. 63/224,923, filed on Jul. 23, 2021.

(51) Int. Cl. B01L 3/00

(52) U.S. Cl. CPC B01L 3/502784; B01L 3/5023; B01L 2200/0673; B01L 2300/0645; B01L 2300/126; B01L 2400/0415

(57) ABSTRACT

Device for concentration of bioparticles for identification comprises one or more capillary flow paths from at least one inlet for advection along the path of bioparticles in a buffer solution, two or more membranes along the flow path, the membranes being individually selectable for electrical powering, thereby to controllably set up a powered membrane region at a location along said path, said powered membrane region causing localized concentration of the bioparticles, digital-like manipulation of the preconcentrated bioparticles plugs, and detection surface immobilized molecular probes located along said flow path to detect the bioparticles following localized concentration.

### RELATED APPLICATIONS

This application is a Continuation of PCT Patent Application No. PCT/IL2022/050797 having International filing date of Jul. 24, 2022 which claims the benefit of priority under 35 USC § 119(e) of U.S. Provisional Patent Application Nos. 63/252,667, filed on Oct. 6, 2021 and 63/224,923, filed on Jul. 23, 2021. The contents of the above applications are all incorporated by reference as if fully set forth herein in their entirety.

### FIELD AND BACKGROUND OF THE INVENTION

The present invention, in some embodiments thereof, relates to a digital microfluidics-like manipulation of electrokinetically preconcentrated bioparticle/biomolecule plugs in continuous-flow, and, more particularly, but not exclusively, to such a device for concentration of biological particles/molecules for identification.

The unique ion permselectivity of ion exchange membranes, stemming from the charged surface groups, predominantly allows counterions to pass through unimpeded while excluding co-ions. Under non-equilibrium conditions, i.e. application of an external field, this symmetry-breaking phenomenon results in the formation of ion-depleted and ion-enriched layers at opposite membrane-electrolyte interfaces, a phenomenon known as concentration-polarization (CP). The ability to induce regions of high and low ionic concentrations adjacent to a permselective membrane or a nanochannel subject to CP has been the focus of intensive research in the last decade, particularly regarding its relation to microfluidic applications, e.g., on-chip desalination where the CP layer is used to separate between brine and desalted streams, or enhanced biosensing by preconcentration of analytes at the edge of the depletion layer. Among various applications, CP-based preconcentration, occurring at the outer edge of the depletion layer due to counteracting convective versus electromigrative fluxes of the third species, is very promising for highly sensitive biosensing as it brings to several orders of magnitude preconcentration of an analyte of interest. In recent years, various microfluidic preconcentration systems using various ion permselective media, such as nanochannels, porous membranes, paper and polyelectrolytic gels, have been investigated for their detection-enhancing capacities.

The main drawback of CP-based preconcentration systems is the inability to control the location of the preconcentrated biomolecule plug and with it an inability to overlap between the preconcentrated plug of target molecules and the surface immobilized antibodies so as to enhance detection sensitivity and binding kinetics. In cases where the antibodies are fixed to the surface of a microchannel, some pre-calibrations are necessary to ensure this overlap, which is very sensitive to the system parameters (e.g., flow rate, voltage, channel geometry etc.). One approach to achieve direct control over the length of the depletion layer, which in turn controls the location of the preconcentrated plug, uses either embedded electrodes or heaters for local stirring of the fluid, via either alternating-current-electro-osmosis (ACEO) or electro-thermal (ET) flow, correspondingly. Another approach to control the location of a single preconcentrated plug is to use two ion permselective membranes in series. It has been shown that, in the intermediate region between the two membranes, the enriched layer prevents the propagation of the depletion layer and its corresponding developed preconcentrated molecule plug. Recently, we demonstrated the ability to simultaneously form a series of multiple preconcentrated plugs at desired locations using the same principle, however, the ions permselective membranes were replaced by individually controlled tunable nanochannels, formed by deformable membranes, in series which enables dynamic operation.

### Concentration-Polarization Based Preconcentration of Molecules for Enhanced Biosensing

### The Problem

For multiplex sensing and/or more complicated assays involving more than a single preconcentrated plug of molecules there is a need to perform sophisticated manipulations of several such plugs in a similar manner to that achieved using digital microfluidics of discrete droplets. Previous studies involving the formation of more than a single plug were focused on simultaneous formation of several plugs in parallel channels for enhanced parallelization of the biosensing. Recently, we demonstrated the ability to simultaneously form a series of multiple preconcentrated plugs connected in series within a single long microchannel using an array of tunable nanochannels, formed by deformable membranes. However, these previous studies do not consider: (a) digital like manipulation (e.g. splitting, merging, down and up-stream translocation) of a single and/or multiple plugs of the same preconcentrated molecules; (b) digital manipulations of multiple plugs containing different preconcentrated molecules from samples introduced via separate inlets.

Previously multiple ion-permselective membranes arranged either in parallel or in series within microfluidics channels cannot enable any form of digital like manipulation. While being able to simultaneously form, pre-concentrated plugs enhance parallelization but cannot enable programmable manipulation of such plugs to perform sophisticated multiplex sensing and other assays involving either a single or multiple inlets into which different samples are introduced. Accordingly, digital like operations on preconcentrated plugs from a single or multiple sample inlets are not possible.

### SUMMARY OF THE INVENTION

The present embodiments may involve extending a single pair of membranes into an array of individually addressable membranes within either a one- or a two-dimensional microfluidic network, that may for example be fabricated or may be a paper-based lateral flow assay. In addition to this structural extension we show how it is possible to operate these in a controlled manner so as to perform digital-like microfluidic operations on preconcentrated plugs, in particular including operations that cannot be realized using a single pair, e.g. down and up-stream translations, splitting, merging, parallelization, multiplex sensing etc.

The term ‘microfluidic network’ may refer to a specifically constructed substance with microtubules for transport of liquid, a capillary flow path, or it may refer to paper that allows for transport of liquid, typically by capillary action.

The operations may be carried out on bioparticles that require identification, in order to concentrate the bioparticles so that identification may be easier. For example a test may look for antibodies to a particular virus, and it is useful to concentrate the biological sample so that the test may find more of the antibodies. The particles may concentrate in plugs, as explained above, which plugs may be manipulated in various ways, including by simply holding the plug for a preset time in the detection region so that the detector particles may do their work.

According to an aspect of some embodiments of the present invention there is provided a device for concentration of bioparticles for identification comprising:
at least one capillary flow path from an inlet for advection of the bioparticles in a buffer solution;
at least one pair of membranes along the flow path, the membranes being individually selectable for electrical powering, thereby to controllably set up a region subject to a voltage gradient, at a location along the path, the region causing localized concentration of the bioparticles into at least one preconcentrated bioparticles plug;
detection surface immobilized molecular probes located along the flow path to detect the bioparticles following the localized concentration.

In an embodiment, the device is a paper-based lateral flow device having at least one test line, and configured such that the localized concentration occurs at the test line

In an alternative embodiment, the device is a concentrator, configured with an outlet with the localized concentration, the outlet configured for extracting the localized concentration of bioparticles when the outlet is aligned to the inlet of a lateral flow device.

In an embodiment, the flow path comprises a microfluidic network of microfluidic channels.

In an embodiment, the at least two membranes comprise an array of membranes, each membrane being individually selectable for electrical powering.

The device may be configured such that changing a selection of powered membranes in the array maneuvers the localized concentration.

In an embodiment, the flow path comprises a one-dimensional path and the membranes are arrayed on the path.

In an embodiment, the flow path comprises a two-dimensional network and the membranes are arrayed over the two-dimensional network.

In an embodiment, the membranes comprise ion-permselective membranes.

In an embodiment, the flow path comprises a paper-based lateral flow assay.

The device may be configured such that the selecting of electrical powering on the membranes performs digital-like microfluidic operations on the at least one preconcentrated bioparticles plug.

In embodiments, the digital-like microfluidic operations comprise one or more of: down and up-stream translations, splitting, merging, parallelization, and multiplex sensing of the at least one preconcentrated bioparticles plug.

The device may be configured to allow digital-like manipulations of multiple plugs containing different preconcentrated particles/molecules from samples introduced via separate inlets

The device may comprise individual electrodes to respective ones of the membranes, therethrough to selectively electrically power the membranes.

The device may differentially electrify the membranes to generate either an enrichment layer or a depletion layer.

Upon the application of a voltage drop between two of the membranes in the face of background net flow in the microchannel, a depletion layer may be generated from the interface of the downstream membrane, thereby to cause the concentration localization to occur at an edge of the depletion layer.

The device may comprise a serial array of at least three individually addressable membranes, and intermembrane spacings, embedded within a respective straight flow path.

According to a second aspect of the present invention there is provided a method of identifying bioparticles/biomolecules in a buffer fluid, comprising:
inserting the bioparticles/biomolecules and buffer fluid into a microfluidic network, the microfluidic network forming at least one capillary flow path;
differentially electrifying individually addressable membranes embedded into the flow path of the microfluidic network, to cause a concentration of the bioparticles/biomolecules into a first localized concentration plug; and
changing the electrifying of the membranes to hold or maneuver the concentration plugs around detection molecular probes.

The device may carry out further changing of the electrifying to generate at least one additional concentrated plug.

The method may involve changing which membranes are electrified to carry out digital-like manipulation of the first and the at least one additional plug.

In the method, the digital-like manipulation may comprise one or more of: down and up-stream translations, splitting, merging, parallelization, holding at a predetermined location for a preset time, and multiplex sensing.

In embodiments, the microfluidic channels comprise fabricated microchannels or a paper-based lateral flow assay.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are simplified schematic illustrations of concentration of bioparticles in depletion regions along flow paths according to an embodiment of the present invention;

FIGS. 2A and 2B are schematic views of formation and manipulations of plugs by electrifying membranes, alongside electron micrographs of the plugs so manipulated;

FIG. 3 is a view of a plug being formed between two membranes according to embodiments of the present invention;

FIGS. 4A and 4B are views of an embodiment of the present invention incorporated into a lateral flow test with test and control lines;

FIGS. 5A and 5B are further schematic views of formation and manipulations of plugs by electrifying membranes, alongside electron micrographs of the plugs so manipulated;

FIGS. 6A-6C are views showing an array of four membranes and different ways of electrifying the four membranes with results according to embodiments of the present invention;

FIGS. 7A to 7E show a microscope image of the fabricated 2D membrane array device depicting eight membranes embedded within the cross main microchannel and connected to side chambers where electrodes are inserted, transient superposed fluorescent microscope images with the manipulation of different locations of preconcentrated plug as by eight digitally controlled serial membranes, transient fluorescent microscope images with manipulation of two different preconcentrated biomolecules, and accumulation and separation of mixed biomolecules (GFP, RFP, and Dylight 488 fluorescent molecules) at the crossing, all according to embodiments of the present invention;

FIGS. 8A and 8B show valving of two and three species respectively, firstly schematically and then as an electron micrograph;

FIG. 9 is a simplified schematic diagram and associated micrograph showing valving using a Yshaped channel according to embodiments of the present invention;

FIGS. 10A and 10B are a simplified view and an exploded view of a device for lateral flow tests having individually powered nafion membranes according to embodiments of the present invention;

FIGS. 11A to 11C illustrate the internal structure and workings of a typical lateral flow test device;

FIGS. 12A and 12B illustrate regions of sensitivity for various tests;

FIGS. 13A to 13C are views of the structure, operation and plug formation in a lateral flow test modified according to the present embodiments;

FIG. 14 is a schematic diagram illustrating suitability of various fluids for testing according to the present embodiments;

FIGS. 15A and 15B show structures for on-strip and off-strip concentration of biological test particles according to embodiments of the present invention;

FIGS. 16A and 16B illustrate experimental setups for a device according to the present embodiments and associated concentrated plugs;

FIGS. 17A and 17B schematically illustrate an off-strip concentrator according to embodiments of the present invention;

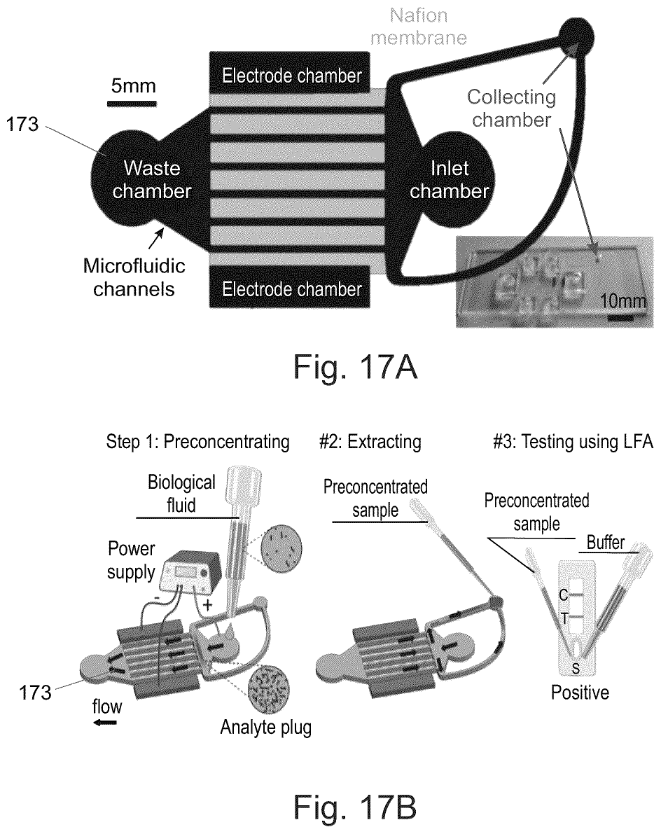

FIGS. 18A-18C illustrate operation of the concentrator of FIGS. 17A and 17B;

FIGS. 19A and 19B illustrate experimental setups for a test device according to the present embodiments; and

FIGS. 20A and 20B illustrate an example of separation between concentrations of different bioparticles due to their different electromigrative mobility, according to embodiments of the present invention.

### DESCRIPTION OF SPECIFIC EMBODIMENTS OF THE INVENTION

The present embodiments may provide a device for concentration of bioparticles for identification comprises one or more flow paths from an inlet for diffusion along the path of bioparticles in a buffer solution, two or more membranes along the flow path, the membranes being individually selectable for charging, thereby to controllably set up a charged region at a location along said path, said charged region causing localized concentration of the bioparticles, and detection molecules located along said flow path to detect the bioparticles following localized concentration.

The present invention, in further embodiments thereof, relates to a digital microfluidics-like manipulation of electrokinetically preconcentrated bioparticle/biomolecule plugs in continuous-flow, and, more particularly, but not exclusively, to such a device for concentration of bioparticles/biomolecules for identification, for example for the purpose of diagnosis.

The present embodiments may provide digital microfluidics-like manipulations of preconcentrated biomolecule plugs within a continuous flow that is different from the commonly known digital microfluidics involving discrete (i.e. droplets) media. This is realized using one- and two-dimensional arrays of individually addressable ion-permselective membranes with interconnecting microfluidic channels. The location of powered electrodes dictates which of the membranes are active and generates either enrichment or depletion diffusion layers, which, in turn, control the location of the preconcentrated plug. An array of such powered/activated (i.e. passing current through) membranes enables formation of multiple preconcentrated plugs of the same biosample as well as of preconcentrated plugs of multiple biosample types introduced via different inlets in a selective manner.

More particularly, for the electrothermal-based control of the location of the preconcentrated plug, a device with an array of individually addressable Nafion membranes embedded on the bottom of the microchannels was fabricated. By controlling the location of powered electrodes it becomes possible to control which of the membranes are active and generates either an enrichment or a depletion diffusion layer, which in turn controls the location of the preconcentrated plug. An array of such powered membranes enables multiple preconcentrated plugs, which is of importance for parallelization. Moreover, digital-microfluidics operations such as up-down and left-right translation, merging, and splitting, can be realized, but on preconcentrated biomolecule plugs instead of on discrete droplets. This technology, based on nanoscale electrokinetics of ion transport through permselective medium, opens opportunities for smart and programmable digital-like manipulations of preconcentrated biological particle plugs for various on-chip biological applications.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Overview of the Conceptual Idea of Digital-Like Operations on Preconcentrated Biomolecule Plugs

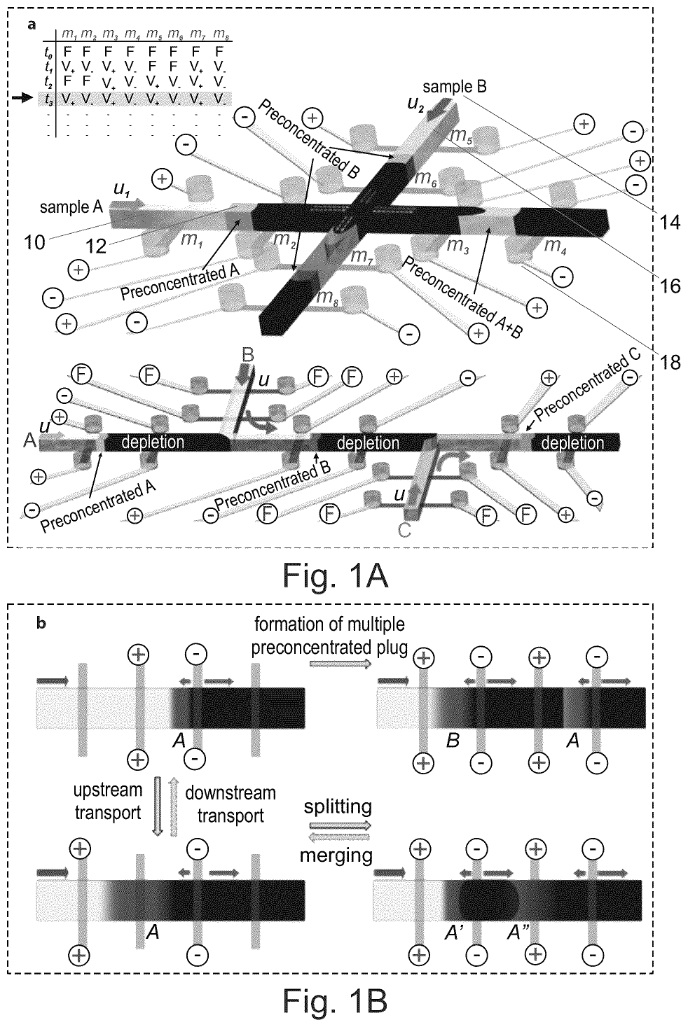

Reference is now made to FIGS. 1A and 1B which schematically illustrate at a conceptual level, digital microfluidics-like manipulation of multiple preconcentrated plugs of a biosample using electrokinetically driven ion concentration polarization (CP) within continuous-flow conditions according to embodiments of the present invention. More particularly, FIGS. 1A and 1B show digital control, that is translation, splitting and merging, of a single preconcentrated plug within a 1D channel geometry.

FIG. 1A shows use of a 2D microchannel and T-shape channel geometries and FIG. 1B gives a simplified schematic explanation of the different digital-like operations on a single preconcentrated plug.

A sample A is introduced at 10 at the end of a linear path 12, and a sample B is introduced at 14 at the end of a linear path 16. Various membranes 18 are located along the paths and are separately electrified as positive, negative or neutral. Thus regions of voltage drop may be set up between adjacent pairs of membranes.

The application of a voltage drop, or more generally a region subject to a voltage gradient, between two membranes embedded within the microchannel with background net flow (e.g. pressure driven, electroosmosis) where a depletion layer is generated from the interface of the downstream membrane results in the formation of a preconcentrated plug in between them. For a downstream cation-exchange membrane (e.g. Nafion) that is negatively biased (i.e. cathode) the field-gradient-focusing effect resulting by counteracting advection and electromigration is responsible for the preconcentration of the bioparticles at the edge of the depletion layer. Since the upstream membrane is oppositely biased (i.e. anode) an ionic enriched layer is generated at its interface, thereby restricting the depletion layer growth to in between the membranes, which in turn also control the location of the preconcentrated plug. Thus, for a series of membranes the locations of preconcentrated plugs are dynamically controlled by activating the selected pair of membranes on demand (FIG. 1b), where the anode-biased (+), cathode-biased (−) membranes are symbolized as “V₊” and “V₋” respectively, while the non-activated membranes remain floating, indicated by “F”. The conceptual digital-like operations of translation, splitting and merging are realized by manipulating a single preconcentrated plug under continuous flow in a simple 1D microchannel geometry as shown in FIG. 1B.

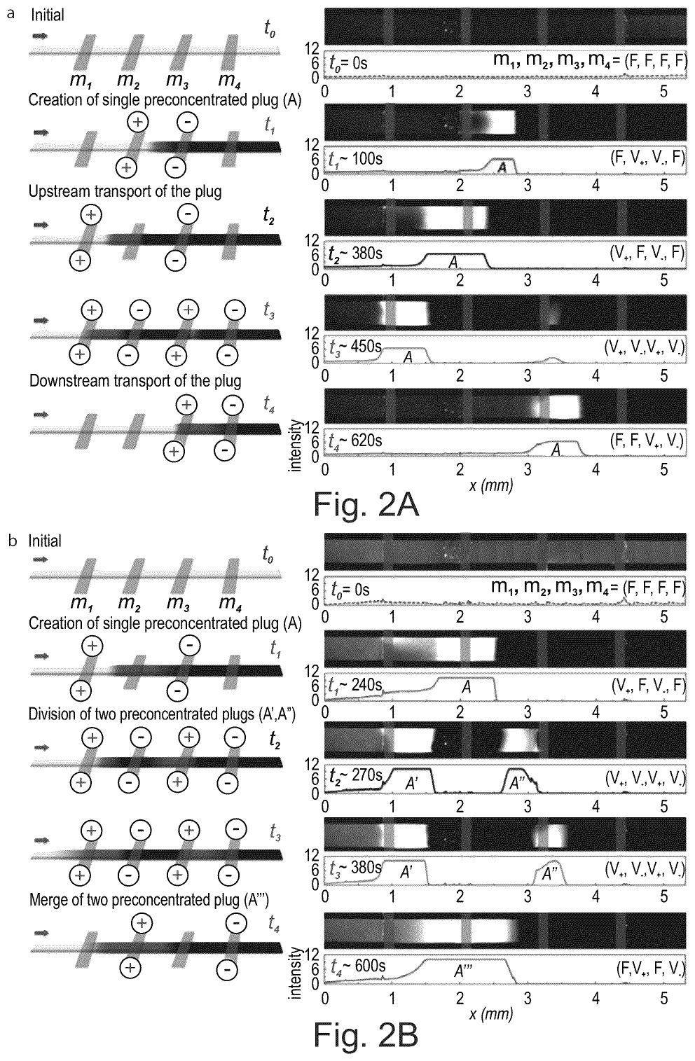

Reference is now made to FIGS. 2A and 2B, which further illustrate the application of digital control operations such as translation, splitting and merging, of a single preconcentrated plug within a 1D channel geometry. FIG. 2A shows upstream and downstream transport of a single preconcentrated molecule plug (A). FIG. 2B shows splitting and merging of a single preconcentrated plug (A into A′ and A″). These time evolving scenarios are presented via a schematic description (shown on the left) along with accompanying fluorescent microscope images (shown on the right) and corresponding normalized (by their initial intensity value) fluorescent intensity profiles. The blue arrow indicates the flow direction and “+” and “−” signs indicate the applied anodic (V₊) and cathodic voltages (V₋), respectively. Times t₁, t₂, t₃, t₄, t₅ represent the change of operation conditions and resulting effect. The distance between two membranes is 1 mm.

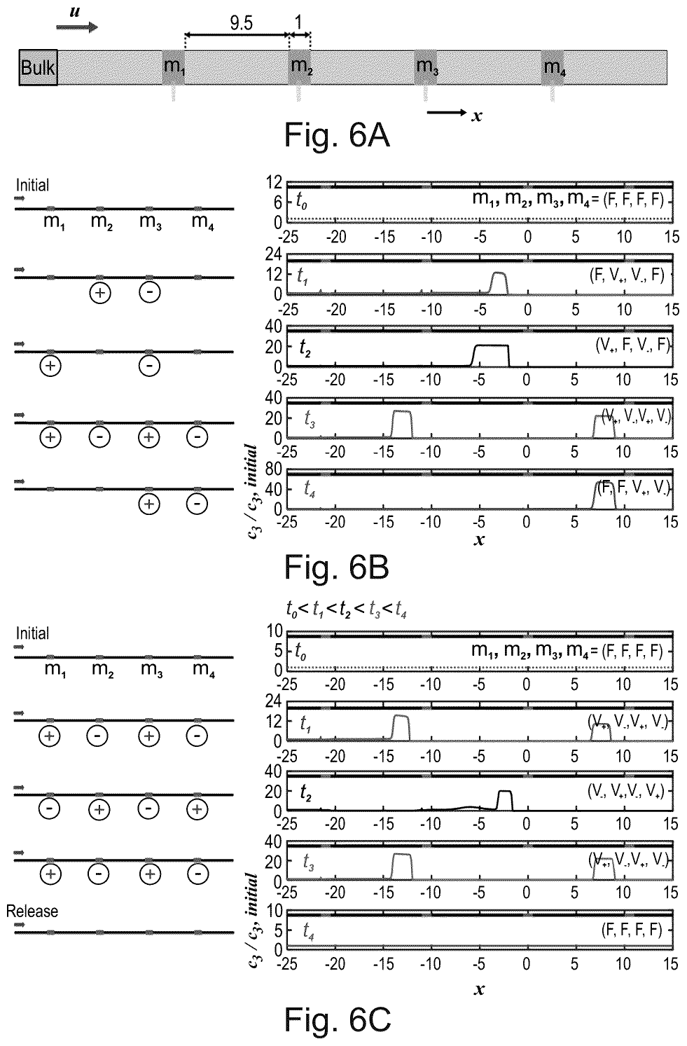

To realize digital-like control of biomolecule plugs we fabricated a serial array of individually addressable four membranes, with 1 mm spacing, embedded within a straight microchannel as discussed above in respect of FIGS. 2A and 2B. FIG. 2A shows the on-demand formation of the preconcentrated plug followed by its upstream and downstream translation by controlling the potential bias of the different membranes under operating conditions of u=99±17 μm s⁻¹ (Pe˜1090) and V=15V₋ The initial equilibrium location of the preconcentrated plug, A was formed between membranes 2 (m₂) and 3 (m₃) by activating only the two membranes (m₁, m₂, m₃, m₄=F, V₊, V₋, F) at t₁. For efficient upstream transport of the preconcentrated plug, two steps of transient operations were needed as depicted in t₂ and t₃ of FIG. 2B. First, the anode-biased membrane (V₊) was switched to upstream membrane (m₁) from the equilibrium condition, while the activated membrane (m₂) was deactivated, that is left floating (V₊, F, V₋, F) which resulted in further upstream propagation of the preconcentrated plug beyond the deactivated membrane (m₂), due to the relatively strong electromigration. Once the preconcentrated plug was passed over the membrane at t₂, the floating membrane was biased with a cathode (V₋) for further upstream transport between membrane 1 and 2, while the downstream-escaped fluorescent molecules were trapped on the next pair of the activated membranes by applying the mode of (V₊, V₋, V₊, V₋) at t₃. The downstream transport of preconcentrated plug (t₄) was relatively easily obtained by simply turning off the upstream pair of activated membranes (F, F, V₊, V₋), mainly due to advection. The experimental results stand in qualitative agreement with the numerical simulations depicting the time-dependent concentration of a third species (c₃), normalized by its initial value (FIG. 6b).

The splitting of a preconcentrated plug (A) was achieved (FIG. 1A, 2B) by forming a preconcentrated plug above a floating membrane under a net flow of u=51±7 μm s⁻¹ (Pe˜304) and V=15V under membrane potential bias of (V₊, F, V₋, F). By changing the floating membrane (m₂) to a cathode-biased membrane with the operation mode of (V₊, V₋, V₊, V₋), the formed preconcentrated plug was divided into upstream (A′) and downstream (A″) preconcentrated plugs at t₂ and t₃. The re-merging of the two divided concentrated plugs was achieved by (F, V₊, F, V₋) at t₄. It is noted that the fluorescent molecules were continuously accumulated at the upstream preconcentrated plug, resulting in an increased fluorescent intensity and widening of the plug within increasing operation time. These results clearly demonstrate that the location of the preconcentrated plug can be digitally and dynamically controlled by controlling the bias of the membranes within the array.

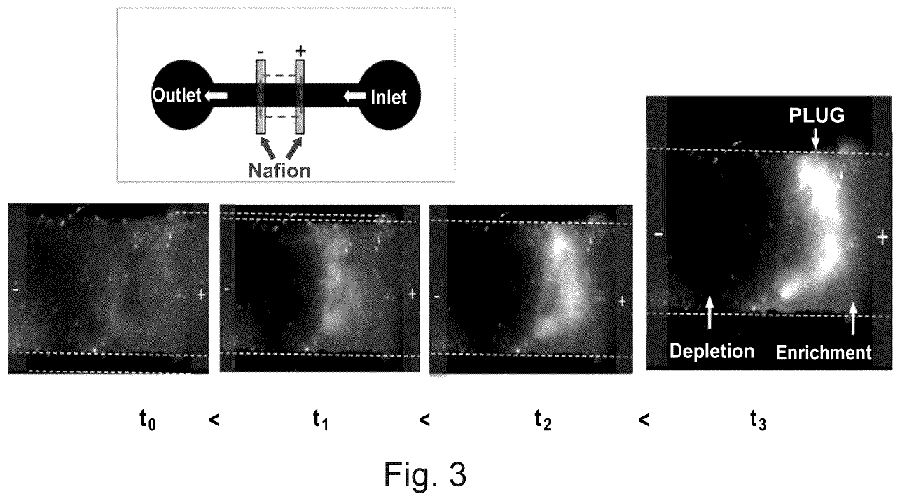

In addition, the ability to form a preconcentrated plug in between two Nafion membranes within a paper-based lateral flow device was demonstrated, and reference is now made to FIG. 3, which shows generation of a preconcentrated molecule plug on a paper-based lateral flow device. CP-based preconcentration of negatively charged fluorescent dye on cellulose filter paper with pore size of ˜11 μm is shown with visualization of the development of a preconcentration plug over time in between two Nafion membranes within a single straight paper-based channel.

In similar manner to a fabricated microfluidic channel, powering the ion perm-selective membranes embedded within the paper-based lateral flow device along with capillary-based advection, enables control of the generation and location of the plug. Following these results, realizing digital control of a single plug within a paper-based device is achievable by adding additional membrane pairs in series.

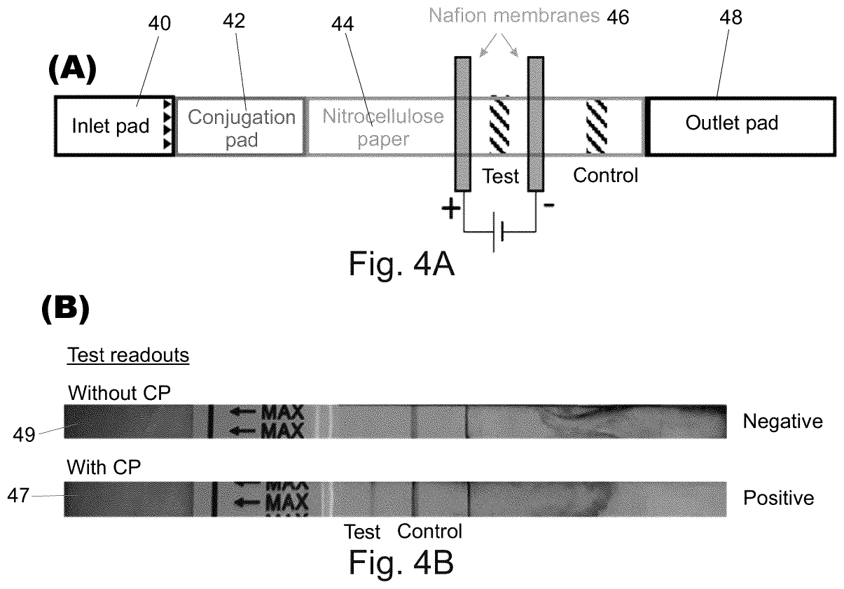

Reference is now made to FIGS. 4A and 4B, which illustrate increasing LOD using CP according to the present embodiments on a commercially available LFA for detection of Strep-A. An inlet pad 40 leads to a conjugation pad 42, and nitrocellulose paper 44 on which are two nafion membranes 46, each separately electrified, that define a test line. A control line lies further down and then an outlet pad 48 is provided. Test readouts with 47 and without 49 particle concentration are shown. Using this method, we demonstrate enhanced detection sensitivity on a commercial rapid test lateral flow assay (LFA) for detection of Streptococcus pyogenes group A (Strep-A) in throat swab specimens. Generally, a standard LFA contains an inlet pad, conjugation pad with gold (Au)/fluorescent particles, nitrocellulose paper with test and control line, and absorption pad. At the test line, the target sample binds to the immobilized antibodies via a sandwich immunoreaction which causes a color/intensity change of the test line. After performing the test, a negative or positive result is obtained just by visual inspection of the test line. In the present embodiments, Nafion membranes were externally connected on the nitrocellulose paper on both sides of the test line which contains Strep-A immobilized antibodies [FIG. 4A]. After introducing the sample solution to the inlet pad, the membranes were powered to generate CP, however, initially the dry paper could not pass electrical current due to its high resistance. Only once the solution past the conjugation pad and wetted the entire paper up to the test line and the membranes, was the electrical current amplified enough to support stable CP and form a preconcentration plug. As a result of the membrane location, the plug is formed onto the binding area. Besides locally increased target sample concentration above the sensing area, the incubation time between the target analyte and the immobilized antibodies was increased as well, which contributes to an enhanced binding to the immobilized antibodies. To test it, we compared the final readouts of a test with and without application of CP for a target sample concentration below the limit of detection (LOD) [FIG. 4B]. We received negative readout for the test without CP (no visualized test line), while obtaining positive readout (clear visualized test line) for the case of which CP was applied during the test procedure.

FIG. 4A shows a schematic description of a commercial LFA kit integrated with Nafion membranes which are individually electrifiable according to the present embodiments, where the test line is in between the Nafion membranes, and FIG. 4B shows a comparison of Strep-A test readouts for target sample concentration under LOD, without applying CP (top image—no visible line—negative readout) and with applying CP (bottom image—visible line—positive readout). Images were taken after the test was fully completed (˜30 min).

### Digital Control of Multiple Preconcentrated Plug within a 1D Channel Geometry

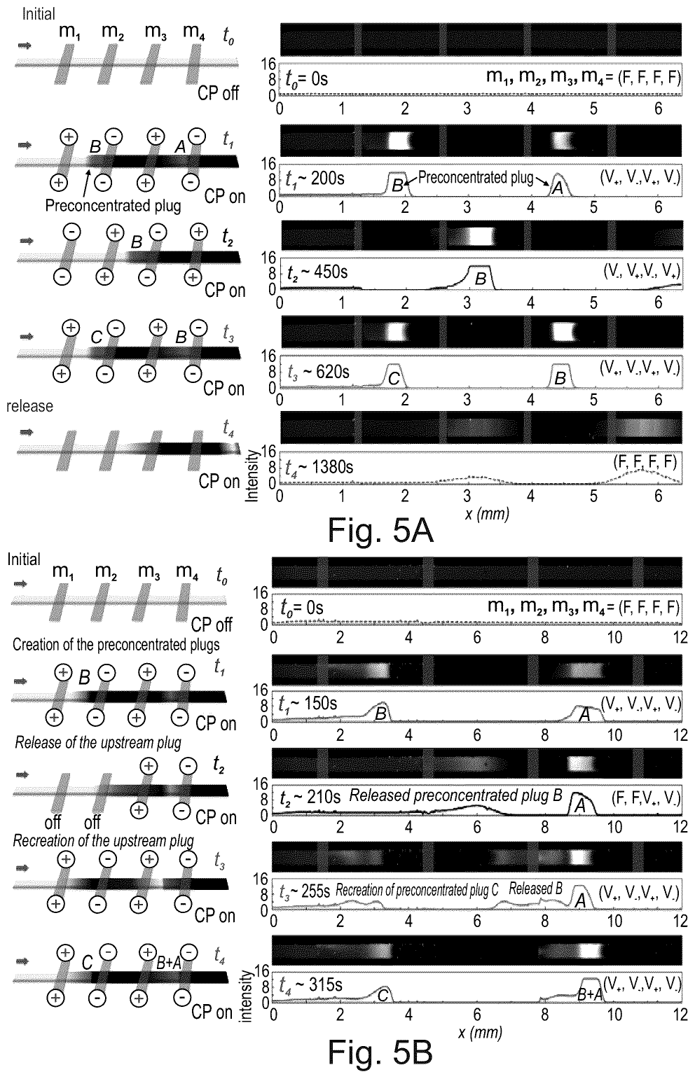

In a further experiment, simultaneous formation and digital control of multiple preconcentrated plugs were examined using a 1D channel with serial membrane array with different spacing (1.8, 2.7 and 1 mm). Such control of multiple plugs is of importance for increasing the parallelization of the system and to enable multiplex sensing when required. Reference is now made to FIGS. 5A and 5B, which show digital control, specifically translation and merging, of multiple preconcentrated plugs in a 1D channel geometry. FIG. 5A shows formation of multiple plugs (A, B, C) and transport to downstream locations. FIG. 5B shows transient generation of multiple preconcentrated plugs (A, B, C) with continuous collection of the upstream preconcentrated molecules at the downstream plugs (A+B). These time evolving scenarios are presented via a schematic description on the left side of the figure along with fluorescent microscope images shown on the right, and corresponding normalized (by their initial intensity value) fluorescent intensity profiles. The blue arrow indicates the flow direction and “+” and “−” signs indicate the applied anodic (V₊) and cathodic voltages (V₋), respectively. Times t₁, t₂, t₃, t₄, t₅ represent the change of operation conditions and resulting effect. The distances between two membranes at a, b are 1.8 and 2.7, respectively.

FIG. 5A shows the simultaneous formation of multiple plugs followed by their translation downstream by periodically switching the operating mode between (V₊, V₋, V₊, V₋) and (V₋, V₊, V₋, V₊) under u=54±7 μm s⁻¹ (Pe˜594) and V=15V). In each cycle, new preconcentrated plugs (C in cycle 2, t₃) are formed. Thus, the number of multiple plugs that are formed and controlled is dictated by the number of activated membrane pairs. An example using three concentrated plugs in a 6-serial membrane array system was also provided. The experimental results stand in qualitative agreement with the numerical simulations depicting the time-dependent concentration of a third species (c₃), normalized by its initial value as shown in FIGS. 6A-C. FIGS. 6A to C show one dimensional numerical simulations of the transient concentration of a third species for upstream and downstream transports according to an embodiment of the present invention. FIG. 6A is a schematic of the 1D model itself. FIG. 6B shows normalized average concentrations of the third species to upstream transport corresponding to FIG. 2A above, and FIG. 6C shows normalized average concentrations of the third species to downstream transport corresponding to FIG. 5A.

An additional scenario that may be provided using digital control of multiple plugs is the merging of preconcentrated plugs and their serial accumulation into a downstream plug. For example, FIG. 5B depicts the transient merging of two preconcentrated plugs (A, B at t₁) after releasing of the upstream preconcentrated plug B to be advected downstream and merge with A between membranes m₃ and m₄. In parallel, a new preconcentrated plug C has formed upstream and needs to be eliminated. This may be realized by simply turning on/off the upstream membrane pair (V₊, V₋, V₊, V₋) to (F, F, V₊, V₋) at t₂ and t₃ with the operating CP conditions (u=159±27 μm s⁻¹ (Pe˜1750)). Here, we only depict a single cycle of their accumulation into a merged plug (A+B), however, accumulation of several such plugs via multiple cycles of operation results in a continuously increased intensity of the fluorescent molecules. We also validate the robustness of this digital control of the multiple preconcentrated plugs by using various geometry (distance between two membranes), and high conductivity (0.01% diluted PBS solution, ˜200 μS cm²).

### Digital Control of Multiple Preconcentrated Plug within a 2D Channel Geometry

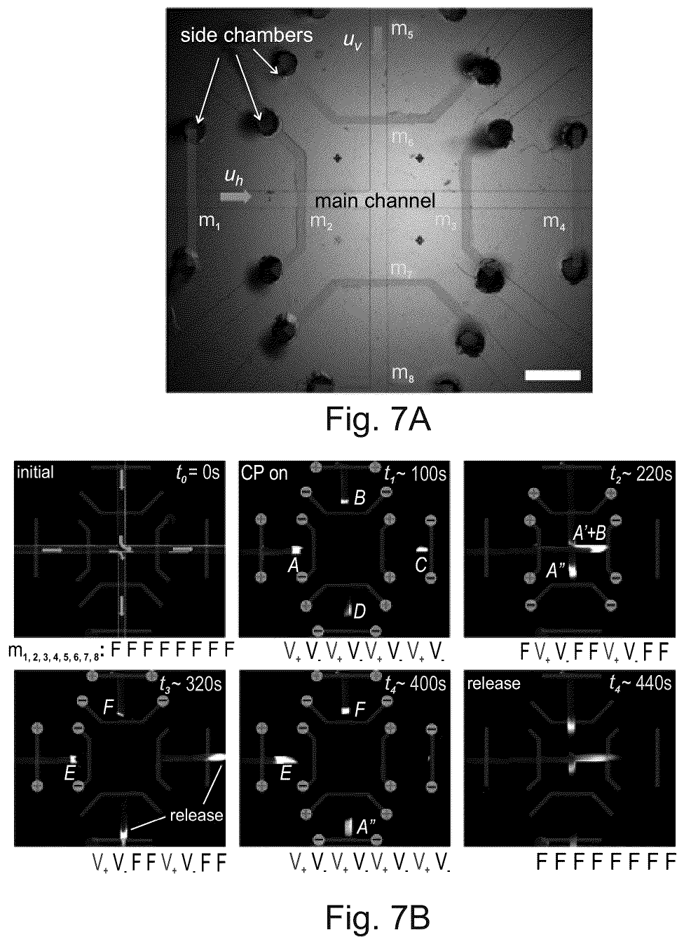

Reference is now made to FIGS. 7A to 7E. FIGS. 7A to 7E show a two-dimensional (2D) microfluidic channel geometry consisting of the crossing of horizontal and vertical channels along with eight membranes that are embedded within it. Thus, enabling the extension of the preconcentrated plug manipulation into a two-dimensional network of fluidic channels for the control of multiple biosmaples that are introduced from different inlets. The separate control over the different flow rates in the horizontal (uₕ) and vertical (uᵥ) channels brings another degree of tunability to the manipulation of the plugs. More particularly, FIGS. 7A to 7E relate to controlling the location of the separation line between brine and dilute streams. FIG. 7A is a microscope image of the fabricated 2D membrane array device depicting eight membranes embedded within the cross main microchannel and connected to side chambers where electrodes are inserted. The serial membranes, m_{1, 2, 3, 4} and m_{5, 6, 7, 8} are located at a horizontal and vertical main channel respectively. Cyan and bright green arrows indicate the horizontal (uₕ) and vertical (uᵥ) net flow respectively. FIG. 7B shows transient superposed fluorescent microscope images with the manipulation of different locations of preconcentrated plug as by eight digitally controlled serial membranes. The horizontal flow is faster than vertical flow (uₕ>uᵥ), and the signal “F” indicates floating the membrane. FIGS. 7C and 7D show transient fluorescent microscope images with manipulation of two different preconcentrated biomolecules (GFP, RFP) in a 2D cross channel. Each schematic illustration and associated fluorescent image at to indicate the initial status where GFP (left, cyan arrow) and RFP (top, green arrow) solutions were introduced with different flow intensity. (White scale bar: 1 mm). FIG. 7E shows accumulation and separation of mixed biomolecules (GFP, RFP, and Dylight 488 fluorescent molecules) at the crossing by generating horizontal and vertical depletion layers from m₃ and m₇ using an operation mode of (F, V₊, V₋, F, F, V₊, V₋, F). The green and red lines in horizontal (x-axis) and vertical (y-axis) graphs indicate the corresponding normalized (by their initial intensity value) fluorescent intensity profiles of GFP with Dylight 488 molecules and RFP respectively.

As a basic scenario, we examined the simultaneous formation of multiple plugs followed by their downstream translation both left to right and top to down by periodically switching the operating mode between (m₁, m₂, m₃, m₄, m₅, m₆, m₇, m₈=V₊, V₋, V₊, V₋, V₊, V₋, V₊, V₋) and (F, V₊, V₋, F, F, V₊, V₋, F) using fluorescent molecules (Dylight 488) as shown in FIG. 7B. Under the operating condition of the applied voltage (15V) where stronger advection exists in the horizontal direction (uₕ>uᵥ), four preconcentrated plugs (A, B, C, D) were generated at t₁. By switching the mode to (F, V₊, V₋, F, F, V₊, V₋, F), the two upstream formed plugs (A, B that are between m₁ and m₂, and m₅ and m₆, respectively) move downstream and accumulate near the crossing area at t₂. Due to the different advection flow rate the horizontal preconcentrated plug (A) is divided between the right (A′) and bottom channels (A″), while the preconcentrated plug at vertical upstream channel (B) is mostly released towards the right channel later and merged into (A′+B) plug. This cycle of the scenario can be robustly repeatable with new preconcentrated plugs (E, F) (t₄ and t₅). Thus, it may show the ability to merge between plugs of molecules from a different source (inlets).

In order to examine the control of preconcentrated plugs from two biosamples, we introduce GFP and RFP fluorescent dyes model molecules, through horizontal and vertical channels under two different conditions of advection (i.e. uₕ<uᵥ and uₕ>uᵥ) as shown in FIGS. 7C and 7D respectively. Here, we mostly activate the membranes embedded on the horizontal channel (m₁, m₂, m₃, m₄) for demonstrating the transient accumulation and release of the plugs. At mode of operation (V₊, V₋, V₊, V₋, F, F, F, F) two horizontal plugs are formed, wherein the upstream is always GFP molecules while the downstream plug consists of either mainly RFP molecules or a mix of RFPs and GFPs depending on the relative flow rates uₕ<uᵥ (FIG. 7C) and uₕ>uᵥ (FIG. 7D), respectively. In addition, the various mixed samples from two inlets (i.e. introduction of GFPs with Dylight fluorescent molecule and RFPs from horizontal and vertical channel) can be entirely accumulated and merged at the crossing by generating depletion layers with horizontal and vertical directions from m₃ and m₇ using the mode of operation (F, V₊, V₋, F, F, V₊, V₋, F) and applied voltage of 40V (FIG. 7E). Also the preconcentrated plug of one sample molecules (e.g, Dylight 488 green fluorescent molecules) can be separated from the merged preconcentrated plug of mixed molecules (e.g., GFPs and RFPs) due to their electrophoretic mobility difference³⁰ again as shown in FIG. 7E, as will be discussed in greater detail below. Those results may demonstrate the extension of digitally controllable preconcentrated plugs in a two-dimensional (2D) channel geometry with multiple inlets and sources of samples.

### Control (Valving) of Preconcentrated Multiple Samples in a Microchannel with Multiple Inlets

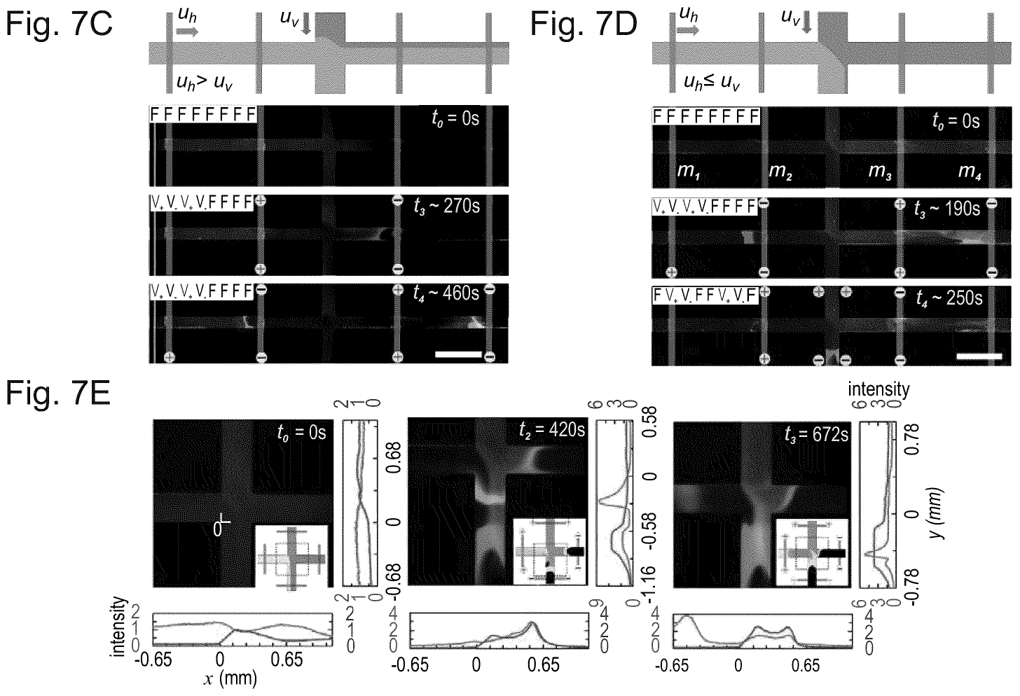

Another approach by serial membrane array operation is to valve the multiple samples from multiple inlets while to preconcentrate the target samples by activating the membrane pairs on demands. For simplicity, we used a microchannel network with two inlets with one outlet and the three embedded membrane pairs to activate preconcentrated plug with valving. The applied voltage for activating CP and net flow of u_{green} and u_{blue} is 25V are 500 nL/min respectively. Reference is now made to FIGS. 8A-B, which shows Control or Valving of preconcentrated plugs of two species in a T-shape microchannel with multiple inlets. These time evolving scenarios are presented via a schematic description (left) along with two superimposed fluorescent microscope images showing blue and green fluorescent molecules (Dylight 405 and Dylight 488) (right) in KCl solution (50 μS cm⁻¹) and corresponding normalized (by their initial intensity value) fluorescent intensity profiles at the main rectangular channel. The green and blue arrow and schematic lines in the graphs indicate direction of flow and the normalized intensity of the blue and green fluorescence molecules respectively.

FIGS. 8A-B clearly show the valving the blue fluorescence molecules at an upstream microchannel (t₁ to t₃) from m₂ while green fluorescent molecules are continuously preconcentrated or released at the downstream microchannel near m₄ and m₆ (t₁ to t₃). Also both fluorescent molecules may accumulate together near m₆ while locations of their fluorescent plug do not significantly overlap due to their slight different electrophoretic mobility (t₄).

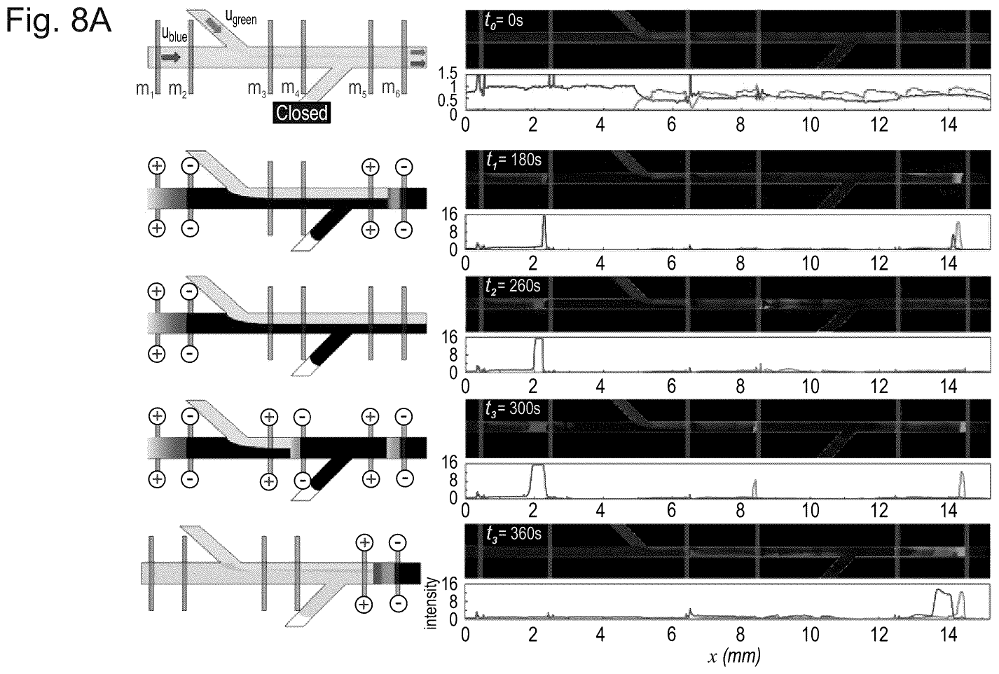

In addition, we use a paper-based Y-shaped channel instead of a fabricated microchannel network, and demonstrate similar valving and preconcentration behavior using an array of Nafion membrane, as shown in FIG. 9. Specifically, FIG. 9 illustrates generation of a preconcentrated plug on a paper-based channel. CP-based preconcentration of negatively charged fluorescent dye is carried out in-situ on cellulose filter paper with pore size of ˜11 μm (Whatman® grade 1). A visualization is shown of the development of a preconcentration plug over time in between two Nafion membranes within an enriched channel (bottom channel) as result of CP-based valving of the top diagonal channel.

We have demonstrated above the realization of an array of individually addressable membranes within a one- and two dimensional network (i.e. cross and multiple inlets) of microchannels that enable digital-like operations such as translation, splitting, and merging of a single preconcentrated plug. These were extended to multiple plugs that can be simultaneously formed and manipulated which is of importance for increased parallelization of the system. The two dimensional array of membranes and microfluidic network add further capabilities that cannot be achieved using a single microfluidic straight channel including the ability to introduce several samples and hence to form plugs of different biosample sources, and control their interaction by overlapping these plugs. This technology permits smart and digital like manipulations of preconcentrated biological particles plugs for various on-chip biological applications.

### Integration of Digital Control of Multiple Preconcentrated Plug to LFA Kit

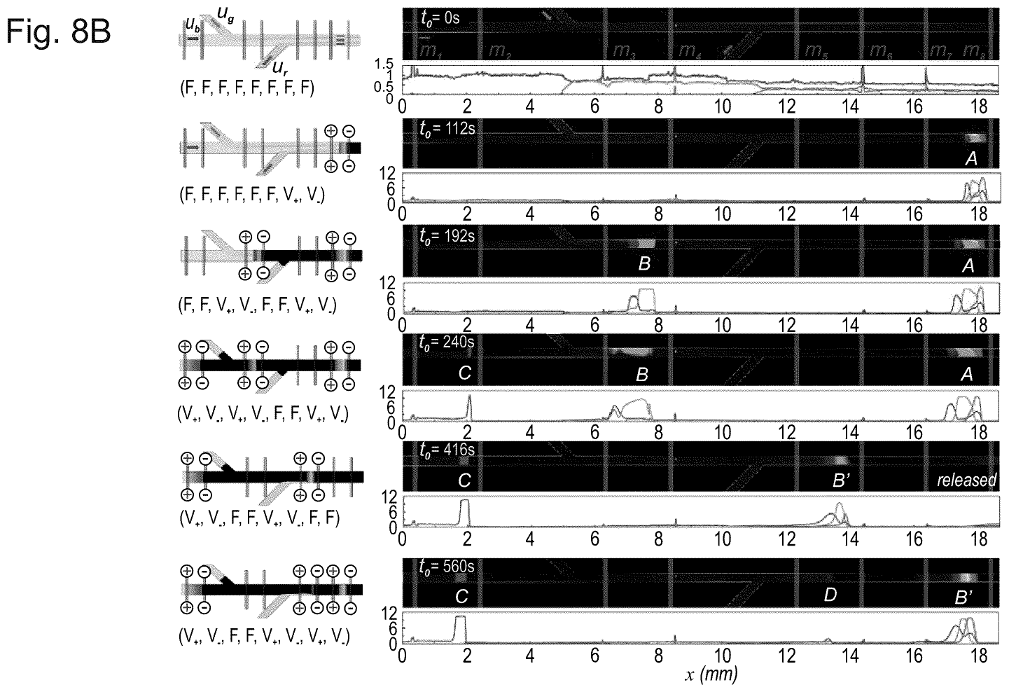

The ability to preconcentrate bioparticles/biomolecules above a sensing area in both a microchannel network and a paper-based channel, in addition to the possibility of forming multiple plugs and performing digital-like operations on the plugs, opens up new capabilities for realization of a hand-held device that can be used for improving detection sensitivity of both specially designed and commercially available LFA kits such as that shown in FIGS. 10A and 10B. The LFA kit may be externally integrated into a box 100 containing individually addressed Nafion membranes 112 and a power supply 113 that lie along the LFA's paper strip of 114. The power supply is located on a supporting printed circuit board (PCB) card 116. The paper is typically for capillary flow, as opposed to chromatography paper which is for separation. The location of each Nafion pair, that is pair of membranes, may be determined according to the location and number of test lines. Feeding the sample solution to the inlet pad, via box inlet 118 and turning on the Nafion membranes in a controlled manner will generate biomolecules plugs that overlap the immobilized antibodies, leading to increased detection sensitivity.

More particularly, FIG. 10A-B is a suggested design of a generic hand-held lateral-flow strip holder that can also use commercially available lateral flow strips and perform programmable operations on the preconcentrated plug of bioparticles, via individually addressable Nafion membranes, for multiplex and enhanced detection sensitivity. FIG. 10A shows a top view of the device consisting of an inlet hole 118, for introduction of the sample and buffer into the paper strip, along with three test lines for multiplex detection, and a control line, and a side view of the assembled device with a partially open top cover. FIG. 10B is an exploded view of all the main system components, including Nafion membranes, test strip, power supply and supporting PCB card.

Materials and Methods

### Experimental Setup

The CP platform design was similar to previously studied open microchannel-Nafion interface devices. In the present embodiments, instead of introducing electrodes within the main microchannel inlets as is commonly realized, the voltage is applied across the membranes embedded within the microchannel. This facilitates the integration of electrodes within the microfluidic system as these are introduced into the side microchannels which are stagnant and hence do not need to support net flow as in the main microchannel where issues such as leakage and introduction of bubbles into the stream is alleviated. The 1D and 2D platform, discussed hereinabove at FIGS. 2A-B and 7A respectively, were comprised of a polydimethysiloxane (PDMS) main microchannel which was 300 μm wide and 22 mm long, 45 μm deep and which had an embedded Nafion membrane array whose dimensions were 300, 200 and 150 μm in width for a distance of 2.7, 1.8 and 1 mm between the membranes respectively. All the membranes were 1 mm long. The membranes interconnected the main microchannel, both 1D and 2D, and side chambers, which were of 1 mm diameter. Instead of inserting the platinum electrodes (0.5 mm-diameter) or embedded electrodes into the microchannel inlets we have connected these to the side chambers of the Nafion membranes. The electrodes were then connected to a hand-made interface unit including a switch array which was individually accessible to the electric signal of V₊, V₋ from a voltage source (Keithley 2636). In order to ensure good contact between the membranes and the paper, wetting and cleaning was required prior to experiments.

For visualization of ionic concentration profiles by CP, 5 μM pH-free Dylight molecules (Dylight 488, Thermo Scientific) were mixed in a 23 μM KCl solution of measured pH 5.3 and conductivity of 3.5 μS cm⁻¹. The fluorescence intensity of the molecules was further analyzed by normalizing the local fluorescent dye intensity by that of the initial intensity before electric field application. In addition, a Green fluorescent protein (GFP, Recombinant A. Victoria GFP protein, abeam) and a Red fluorescent protein (RFP, Recombinant RFP protein, abeam) were diluted to concentrations of 100 ug/mL in in 0.01% diluted PBS (σ=180 μS cm⁻¹) for visualizing the simultaneous manipulation of two preconcentrated molecule plugs with different fluorescence excitation/emission wavelengths in a 2D channel geometry system. The net flow was driven by hydraulic pressure difference between microchannel inlets. All experiments were recorded with a spinning disc confocal system (Yokogawa CSU-X1) connected to an inverted microscope (Eclipse Ti-U, Nikon) and a camera (Andor iXon3). The fluorescence intensity of the molecules was further analyzed by normalizing the local fluorescent dye intensity by that of the initial intensity before electric field application.

### Numerical Simulations

The experimental observations were complemented by numerical simulations for a qualitive comparison. To that end, a simplified one-dimensional (1D) system comprised of a series of membranes (four membranes) that are embedded into a straight long microchannel as shown in FIG. 6A above. The 1D time dependent ion transport is governed by the dimensionless Nernst-Planck-Poisson equations (tilde notations are used below for the dimensional variables, as opposed to their untilded dimensionless counterparts):

$$\begin{matrix}\frac{\partial {c}_{i}}{\partial t} = - \frac{\partial {j}_{i}}{\partial x} = {D}_{i} \frac{{\partial }^{2} {c}_{i}}{\partial {x}^{2}} + {z}_{i} {D}_{i} \frac{\partial }{\partial x} ( {c}_{i} \frac{\partial φ}{\partial x} ) - Pe \frac{\partial {c}_{i}}{\partial x} , i = 1 , 2 , 3 , & ( 1 ) \\ - 2 {ε}^{2} \frac{{\partial }^{2} φ}{\partial {x}^{2}} = {\sum }_{i = 1}^{3} {z}_{i} {c}_{i} - N , & ( 2 )\end{matrix}$$

where Eq. (1) is the Nernst-Planck equations satisfying the continuity of ionic fluxes. For convenience, we use i=1 for K⁺, i=2 for Cl⁻ and i=3 for negatively charged third species. zᵢ and Dᵢ are the valence and the dimensionless diffusion coefficient (normalized by the diffusion coefficient of K⁺, \tilde{D}₁) of species i, respectively, where z₁=−z₂=−z₃=1, D₁=B₂=1 and D³=0.2. All ion concentrations \tilde{c}ᵢ were normalized by the concentration of an electrically neutral solution (the outer stirred bulk concentration), \tilde{c}₁=\tilde{c}₂−z₃\tilde{c}₃=\tilde{c}₀. The spatial coordinate \tilde{x} was normalized by the length of the membrane \tilde{L}, the time \tilde{t} was normalized by \tilde{L}²/\tilde{D}₁, and the ionic fluxes \tilde{J}ᵢ was normalized by \tilde{D}₁\tilde{c}₀/\tilde{L}. The dimensionless parameter Pe appearing in Eq. (1) is the Peclet number and is defined as Pe=ũ\tilde{L}/\tilde{D}₁, where ũ is the flow speed. Eq. (2) is the Poisson equation for the electric potential \tilde{φ}, which was normalized to the thermal potential \tilde{R}\tilde{T}/\tilde{F}, where \tilde{R} is the universal gas constant, \tilde{T} is the absolute temperature and \tilde{F} is the Faraday constant. The parameter ε is the dimensionless Debye length and is defined as

$$ε = \frac{{\tilde{λ}}_{D}}{\tilde{L}} = \frac{1}{\tilde{L}} \sqrt{\frac{{\tilde{ε}}_{0} {ε}_{r} ε \tilde{R} \tilde{T}}{2 {\tilde{F}}^{2} {\tilde{c}}_{0}}} , \qquad ( 3 )$$

where \tilde{ε}₀ and εᵣ are, respectively, the permittivity of the vacuum and the relative permittivity. Finally, N is the dimensionless fixed charge density of the membrane, which was assumed N=0 outside the membrane's regions and N>0 within these regions.
At the inlet boundary (left side), the concentrations were fixed equal to the concentrations in the electrically neutral stirred bulk:

$${c}_{1} = 1 , {c}_{2} = 1 + {z}_{3} {\overline{c}}_{3 , 0} , {c}_{3} = {\overline{c}}_{3 , 0} , \frac{\partial φ}{\partial x} = 0 \qquad ( 4 )$$

where c̅_{3,0} is the dimensionless concentration of the third species in the outer stirred bulk. At the outlet boundary (right side), the following boundary conditions were specified:

$$\frac{\partial {c}_{i}}{\partial x} = 0 , \frac{\partial φ}{\partial x} = 0. \qquad ( 5 )$$

Furthermore, potential biases were applied to the middle of the membranes. On demand, according to the desired application, the membranes were activated by applying positive and negative biases or non-activated. The system (1)-(5) was solved numerically, taking equilibrium as an initial condition, for c̅_{3,0}=0.1, δ=10⁻³ Pe=3 (\tilde{L}˜ 0.2 mm, \tilde{D}˜2×10⁻⁹ m²/s, ũ=30 μm/s) V=700 and N=10.

In the present embodiment, the idea is to improve the detection sensitivity of commercially available lateral-flow assays (LFAs) by introducing electrokinetic preconcentration (several order of magnitude) of the target analyte in the ways described above. Such paper-based lab-on-a-chip devices, that are well-known in-home pregnancy tests, have become extremely popular since the outbreak of the COVID-19 pandemic. Although very attractive due to their low-cost, simplicity and rapid operation, LFAs based on paper strips suffer from low detection sensitivity at the early stage, of the disease, when the target biomarker concentration is low. To increase the concentration of the target analyte, the present embodiments may provide both on- and off-strip electrokinetic preconcentration strategies wherein a preconcentrated plug of molecules is formed upon passage or an electric current through an ion-permselective membrane as explained hereinabove. For on-strip applications, the plug may be formed on-the-fly within the strip and spatially located over the test line in order to increase the chance of target biomarkers binding to the surface-immobilized probes For off-strip applications, the biomarker may be preconcentrated by an external microfluidic device integrated into the LFA inlet. Here, we focus on applying these techniques to commercial kits to be used as is, so as to provide a generic solution.

Rapid diagnosis of a disease in its early developmental stages is critical for effective preventive medicine. Lateral-flow-assays (LFAs) are paper-based platforms used in both point-of-care diagnosis and self-testing due to their low-cost, simple operation, and rapid response.

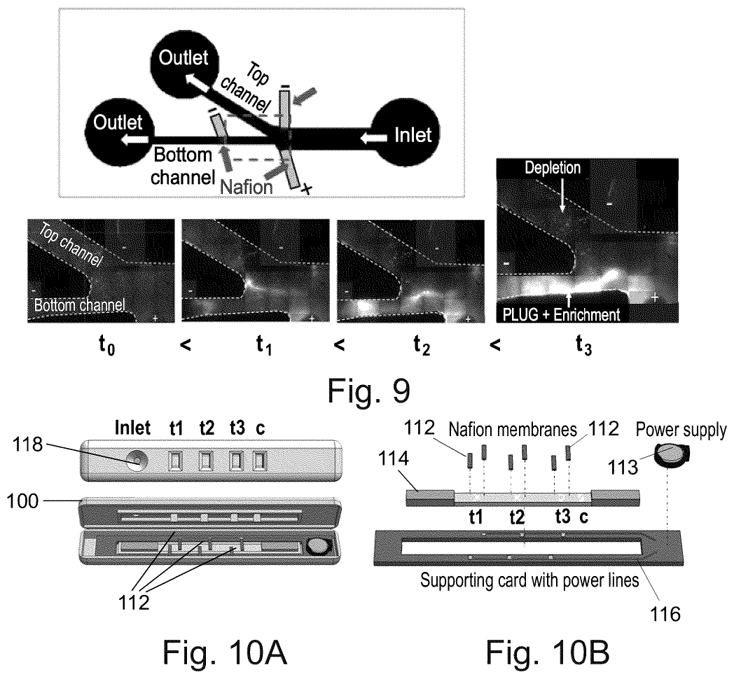

Referring now to FIG. 11A, a typical LFA 110 consists of a sample pad 112, a conjugate pad 114, a reaction membrane 116 containing test 118 and control 120 lines and an absorbent pad 122, wherein the detection mechanism is based on biochemical antigen-antibody interactions. Referring now to FIG. 11B, the biosample (e.g., urine, blood, saliva) is applied onto the sample pad and spontaneously transported via capillary forces along the strip. The target analyte (e.g., protein, virus, bacteria) within the biosample interacts with antibody-conjugated nanoparticles (e.g., gold, silver or carbon nanocolloids²), preloaded in the conjugate pad, as well as with the surface-immobilized capture probes on the test line, yielding a rapid (<30 min) and distinct signal in the form of a colored line. A second line (control) is formed by trapping of excess nanoparticles conjugate, confirming test validity. However, as shown in FIG. 11C, LFA kits suffer from low detection sensitivity as only a small fraction of the target bioparticles binds to the surface-immobilized antibodies due to their rapid passage over the test line, and also lower specificity compared to PCR-based tests due to non-specific absorption, resulting in false-negative and false-positive results, respectively. Consequently, the control of disease transmission (e.g., SARS-CoV-2, influenza and HIV) primarily relies on more complex laboratory tests such as polymerase chain reaction (PCR) and enzyme-linked immunosorbent assay (ELISA) (FIGS. 2A-B), while the contribution of LFAs is limited. This is particularly true in the early stages of the disease when biomarker concentration is low, as was recently demonstrated for COVID-19 detection using LFAs.

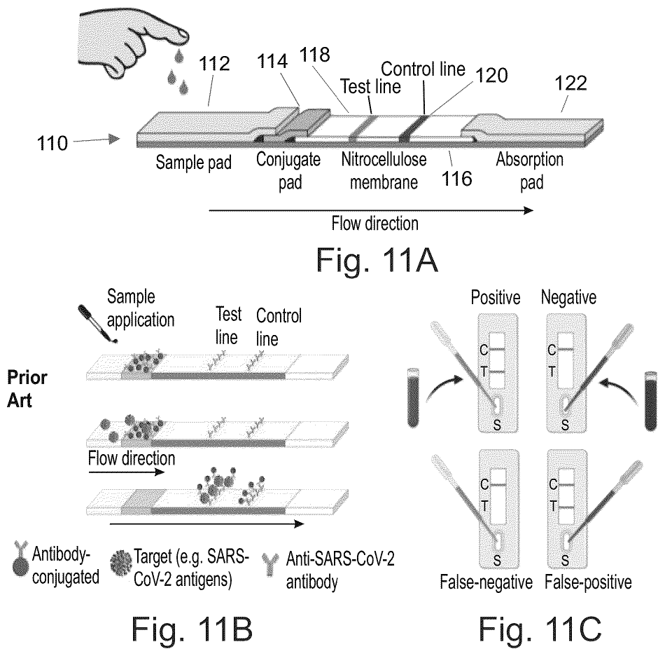

Hence, there is an urgent need to improve the detection sensitivity of LFAs, to facilitate early diagnosis, while maintaining their cost- and time-effectiveness. FIGS. 12A and 12B show the limits of detection in current LFA's against both time and concentration, and indicates regions of concentration in which assistance may help. Also shown is a comparison with other tools. Several approaches have been proposed to address these limitations including: 1) increasing reaction kinetics (e.g. decoupling the delivery of the reporting antibody-conjugated nanoparticles and antigen onto the capture antibody line (sandwich assay)⁴, 2) extending reaction time by slowing the flow rate⁵, 3) signal amplification with high-performance readers/sensor (e.g., optical⁶/electrochemical⁷/magnetic⁸). However, these methods involve physical modifications of the test strip, and additional assay steps, time and cost.

LFAs may also be improved by preconcentrating the target analyte. Effective preconcentration of the target analyte, using both on-strip⁹ and off-strip methods³, may significantly improve the limit of detection of LFA devices, thereby reducing false negative cases say by improved signal-to-noise ratio, and increase detection sensitivity. Various preconcentration methods have been applied to paper-based devices, including magnetic field-assisted separation and electro-kinetic concentration methods (e.g., isotachophoresis (ITP) and ion concentration-polarization (ICP)). ICP-based preconcentration has proven to be an effective and promising sample preconcentration approach due to its robustness compared to ITP which requires leading and trailing buffers.

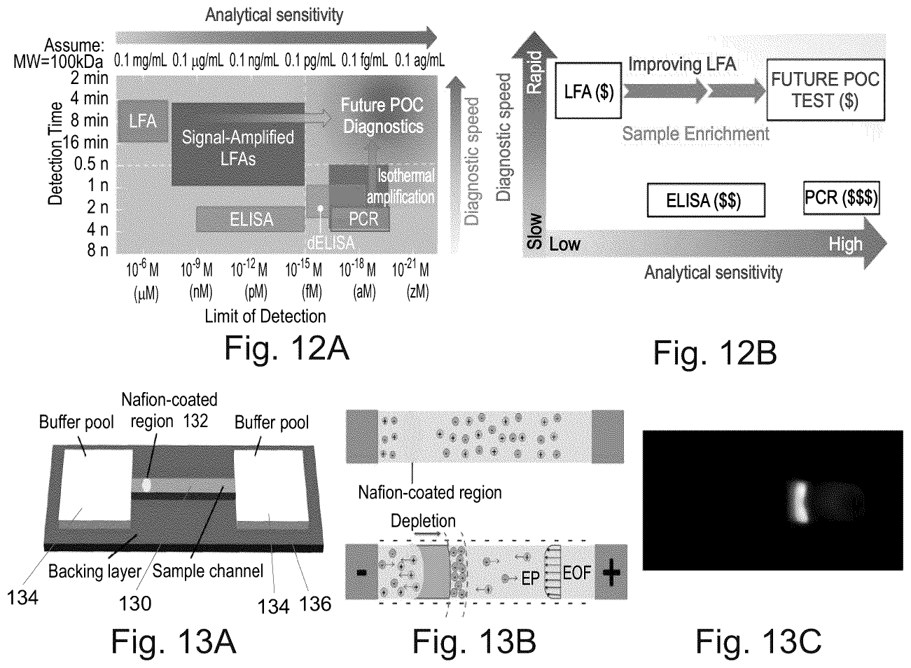

Reference is now made to FIG. 13 (a) which is a schematic showing a paper strip 130 with a Nafion membrane-coated region 132 forming a sample channel between buffer pools 134. The paper is mounted on backing layer 136. FIG. 13 (b) schematically shows depletion around the nafion coated region 132 and shows and specifically electrokinetic preconcentration of analytes upon passage of an electric current through the Nafion. FIG. 13(c) is a microscopic image of a preconcentrated fluorescently labelled molecule plug.

ICP-based preconcentration involves continuous accumulation of charged analytes at a location along an ionic conductivity gradient, that is within the depletion layer that is formed upon passage of a current through an ion-permselective membrane, wherein a force equilibrium between convective and counteracting electromigrative forces exists, an effect also known as field-gradient-focusing. It is possible to integrate the ICP preconcentration approach into a paper strip with patterned Nafion regions and demonstrated preconcentration of several orders of magnitude.

However, integration of an ICP-based preconcentration method into paper strips is far from maturity for implementation into commercial LFAs. The fixed configuration of commercial LFA poses several challenges that need to be addressed. For example, studies testing the method used fluorescent dyes as reporters instead of the commonly used gold nanocolloids. They also used a cellulose paper that enabled clear visualization in contrast to the opaque nitrocellulose membrane used in commercial LFAs. Furthermore, printing of Nafion membranes onto the paper strip or the inlet pad²⁷ is inapplicable if one wishes to use a commercial LFA without modifying the test strip, that is to say if one wishes to use the strip as provided. Other limitations include the failure to assess potential effects of ICP on the gold nanocolloids, which may adversely impact the reaction (e.g., non-specific aggregation/adsorption). Adding external Nafion membranes onto commercial LFA also poses challenges, e.g. ensuring good electrical contact. In addition, the location of the preconcentrated target analyte needs to be controlled to ensure an overlap with the surface-immobilized antibodies for the enhanced binding, due to increased incubation time and local analyte concentration, of preconcentrated antigens to the capturing antibodies on the test line. Such on-strip control has recently been demonstrated via periodic actuation of the ICP, resulting in oscillation of the preconcentrated plug multiple times over the sensing area

Another advantage of on-strip ICP-based preconcentration is the possibility to control an array of individually addressable membranes in accordance with the present embodiments as described herein to enable programmable control of the preconcentrated plug(s) over an array of test lines with varying immobilized antibodies for multiplex sensing. As explained above, programmable control of both single and multiple preconcentrated plugs is disclosed herein.

In a further embodiment of the present invention, off-strip preconcentration overcomes the challenges posed by on-strip operation and can be applied for sufficiently large sample volumes (0.5-1 ml). While several studies demonstrate the usage of microfluidic devices for ICP-based preconcentration achieved by sample volume reduction while simultaneously trapping the target analyte (e.g., radial preconcentrator, they all failed to extract the small volume of preconcentrated molecule plug from within the microchannel without further diluting it. Referring again to FIG. 5A, there is shown digital control of a single preconcentrated plug within a microfluidic channel. Upstream and downstream translation of a single preconcentrated molecule plug A is shown. These time-evolving scenarios are presented via a schematic description (left) along with fluorescence microscope images (right) and corresponding normalized fluorescence intensity profiles. The blue arrow indicates the flow direction and “+” and “−” indicate the applied anodic (V₊) and cathodic voltages (V₋), respectively. The indicated times (seconds) correspond to the change of operation conditions. The white scale bar indicates an area of 1 mm².

Integration of electrokinetic preconcentration with a commercially available LFA into a user-friendly and simple-to-operate device can be inspired by already existing solutions involving integration of LFAs with various add-ons for various purposes. For example, the digital pregnancy test combines electronics, optical detection and display within a single device. A similar integration can be realized for on-strip preconcentration but including membranes for preconcentration. In addition, user-friendly off-strip devices for sample processing (e.g., extraction of the sample from the swab and its mixture with the buffer in one step) with fewer operation steps and human errors, can be found already in several commercially available LFA kits. Hence, a similar end-user experience can be achieved using similar off-strip devices in terms of size and integration with the LFA, for electrokinetic preoconcentration of the analyte in accordance with the present embodiments.

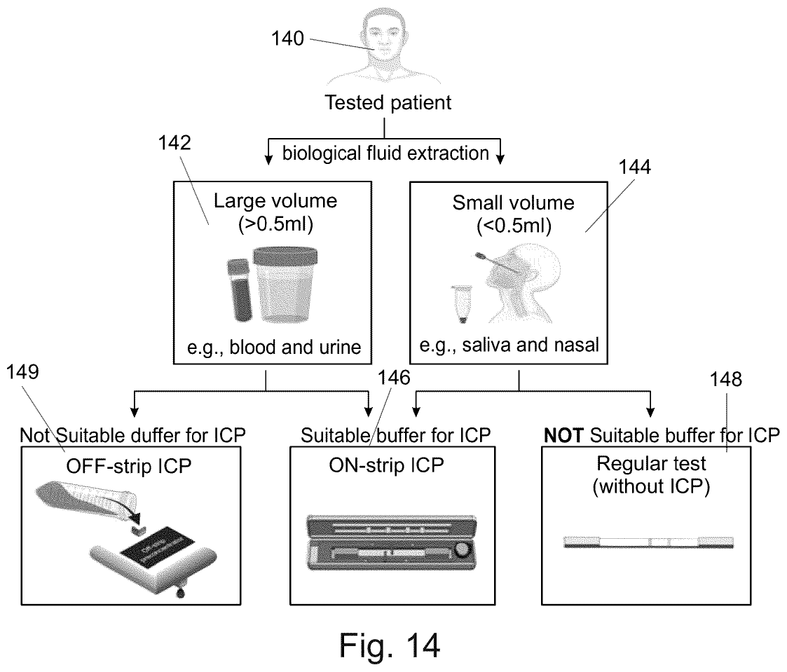

The present embodiments may enhance the detection sensitivity of commercially available LFAs by electrokinetic preconcentration of the target biomarker. Use of the present embodiments may retain the unique advantages of LFAs, i.e. affordability, and simple operation intended for self-testing and time-effectiveness. Specifically, two embodiments are used: (1) on-strip preconcentration of the target biomarker to significantly increase chance of biomarker binding to the surface immobilized probes, (2) off-strip microfluidic ICP-based biomarker preconcentration. Several designs are provided for increased biomarker trapping efficiency with minimum loss as well as their compatibility with the LFA strip for simplified operation. Reference is now made to FIG. 14, which illustrates which approach is suitable for which circumstance. Thus the sample is taken 140 from the tested patient. In some cases, such as blood and urine, the sample is available in quantities in excess of 0.5 ml—142. In other cases, such as saliva and nasal fluids, the typical sample size is less than 0.5 ml, 144. A further complication is that buffer fluids are chosen for their utility in uptake of the substance being tested for by the detecting substances. Not all of these buffers are suitable for ICP. For example some buffers are highly conductive. Accordingly, if the buffer is suitable for ICP, then on strip ICP may be used regardless of the volume—146. If the volume is large and the buffer is not suitable, then off-strip ICP may be used—149. If the volume is small and the buffer is not suitable, then only a regular test without concentration is recommended—148.

More particularly, we suggest various electrokinetic preconcentration approaches which can be divided into on- and off-strip strategies, each strategy with its unique features and advantages. The optimal strategy may depend on several factors, such as sample type (e.g., saliva, urine, and blood) and volume, and buffer. As mentioned, the buffer is designed for efficient analyte extraction and biochemical reaction and is different for different analytes. While the off-strip strategy suits relatively large biological fluid volume, regardless of the buffer type, the on-strip method processes much smaller volumes and requires a buffer that supports ICP for analyte preconcentration.

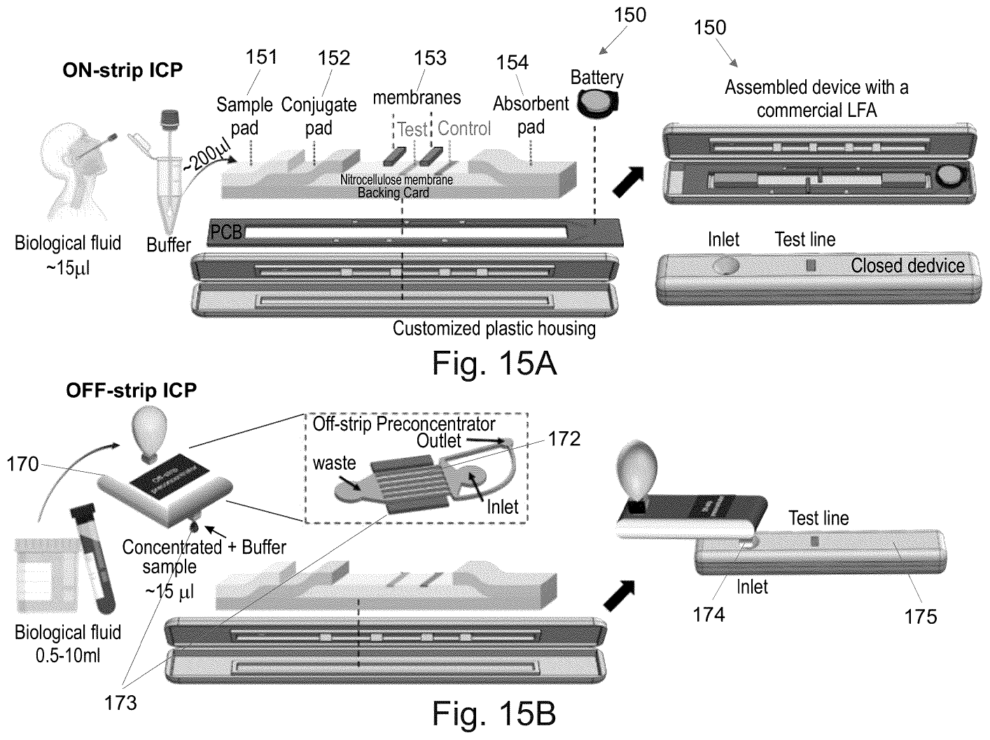

Reference is now made to FIGS. 15A and 15B, which illustrate schematics of on-strip and off-strip platforms designed to improve the sensitivity of commercial LFAs by use of the present embodiments and individual electrification of the Nafion membranes. FIG. 15A shows an on-strip ICP 150 having a sample pad 151, conjugate pad 152, membranes 153 and absorbent pad 154, The Nafion membranes and electronics are integrated externally to the strip within a customized holder 15.

FIG. 15B shows an off-strip microfluidic platform 170, that is integrated into the LFA inlet, for preconcentration of the target analyte through volume reduction of a large sample. Outlet 173 provides the concentrate, which may then be added to the inlet 174 of a typical test device 175.

On-Strip Preconcentration

In the on-strip embodiment of FIG. 15A, the ICP and the corresponding preconcentrated analyte plug are formed within the LFA test strip. The on-strip ICP preconcentrating system may be composed of a dedicated external holder into which the LFA strip is inserted, electronics, and the ionic perm-selective membranes, which are forced to be in contact with the LFA strip when the holder is closed. The present embodiments differ from the prior art LFA strip inter alia in that the membranes are individually electrifiable. As with a standard LFA kit, the solution to be tested is externally prepared (i.e., sample extraction and mixing with the LFA buffer in an external tube) and the obtained mixture is added to the test strip. ICP is then activated by local application of an electric field on the strip itself simultaneously to the transport of the solution mixture via capillary wetting. During the ICP-based on-strip preconcentration process, the preconcentrated plug of target analytes spatially overlaps the test line, resulting in significant enhancement of the binding of the target analytes to both the immobilized capturing probes on the test line and to the reporting nanocolloids due to the increase of both the local analyte concentration as well as the incubation time over the test line. These may enhance analyte detection, without additional requirements from the end-user. The embodiment is suitable based on compatibility of the ICP operation conditions with the LFA reaction buffer as mentioned above, and furthermore, the effect of ICP on the gold nanocolloids, ionic-perm selective membranes and LFA strip contact may need to be checked in individual cases.

Systematic Experimental and Numerical Study to Optimize On-Strip ICP Operation:

To determine the optimal conditions of ICP operation and to examine its effect on the target biomolecules, conjugated nanocolloids, and flow within the test strip, we performed systematic experiments using materials that better represent commercial LFA kits (i.e., nitrocellulose, buffer) instead of previously tested conditions (cellulose, KCl solution). The effect of the porous structure of the paper on ICP development, and particularly, its contribution to analyte plug stabilization and electro-osmotic flow (EOF)-induced flow is investigated numerically for a better physical understanding of the phenomenon.

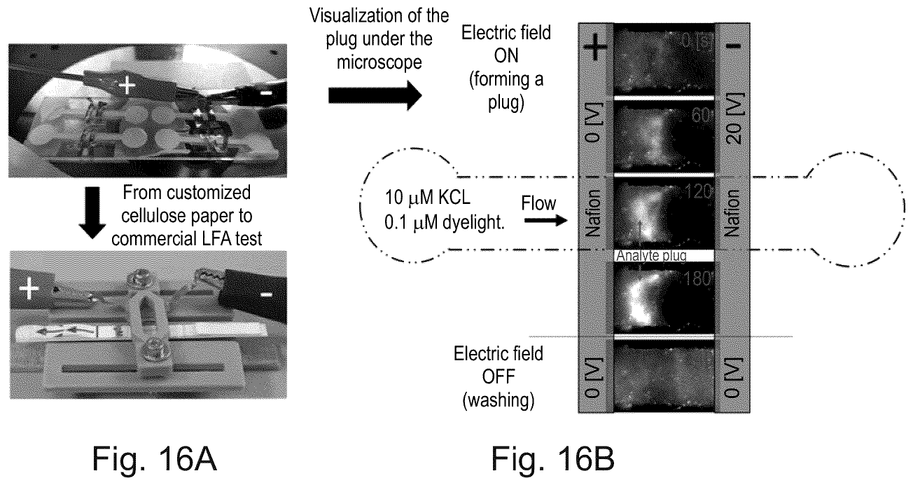

Reference is now made to FIGS. 16A and 16B. Paper-based ICP preconcentration on a cellulose paper strip (Whatman©, grade 1) is shown, where two ionic perm-selective membranes (Nafion©) are externally attached to the paper—See FIG. 1A top. A fluorescent dye molecule (Alexa 488) in a KCl solution (10 μM) is used as a simple target analyte. Application of an electric field through the membranes results in formation of a preconcentrated plug (˜1 mm wide, a preconcentrating factor of ˜20) of fluorescent molecules in between the membranes within 3 min as shown on FIG. 16B. The same approach is applicable to a commercial LFA strip integrating such external membranes, as shown in FIG. 1A bottom. In real LFAs, target molecules are invisible (i.e. non-fluorescently tagged), and their binding is visualized using reporters made of antibodies-conjugated nanocolloids that are preloaded onto the conjugate pad and introduced into the test strip together with the target analyte during its capillary wetting. The electric fields applied for preconcentration of the target molecules may influence these nanocolloids.

More particularly, FIG. 16B shows results of on-strip ICP. ICP-driven preconcentration on a customized cellulose paper as well as with a commercial LFA test. The electric field was applied to copper foils acting as the electrodes through a pair of Nafion membranes, where the membranes act as a cationic perm-selective membranes, and are sandwiched between the paper/LFA strip. The formation of a preconcentrated fluorescent dye (Alexa 488, 0.1 μM in 10 μM KCl electrolyte) plug within the cellulose paper was visualized under the microscope.

Proof of Concept of Enhanced Sensitivity of a Commercially Available LFA:

We examined different methods, such as pressing, penetrating, and layering on commercially available LFAs in order to arrive at an effective contact between the paper and the membrane. A preliminary experiment indicating the enhanced detection signal in an on-strip format of bioparticle concentration was shown above in FIGS. 4A-B.

### Materials and Methods:

Examining ICP efficiency using commercial LFA materials: Materials (e.g., nitrocellulose, buffer) from an available LFA are tested, while their effect on the ICP-induced preconcentrated analyte plug may be monitored by either visualization of fluorescent molecules under a microscope or of the conjugated nanocolloids and immobilized molecular probes.
Examining the ICP effects within a porous media such as chromatography paper and sponges, using numerical simulations (COMSOL Multiphysics).
Target analytes and kits: Mouse IgG, DNA, bacteria (E. coli).

### Risks and Mitigation:

Non-specific absorption and undesired aggregation of the nanocolloids due to the ICP. In such a case, we may in one embodiment introduce the nanocolloids only after the ICP-enhanced binding of the target analytes to the test line is complete.
Inability to visualize plug formation within a commercially available LFA (due to the thicker and denser paper) which makes it difficult to optimize the ICP. In such a case, it is possible to use less dense/thick paper strips that are not used in commercial LFAs for the sake of evaluation of the plug formation.
Extraction/reaction buffer unsuitable (e.g., ionic strength too high) for stable formation of the ICP. Here, we may in one embodiment use a diluted, less conductive buffer that still enables proper biochemical binding. Another embodiment may involve replacement of the buffer with other commercially available less conductive ones. Where possible, no changes are made to the buffer.
Inability to preconcentrate the analyte using electric charge due to its weak/negligible electric charge (operation near the isoelectric point). Here, the pH is the dominating factor and we examine other buffers and/or tweak the pH of the working buffer away from the isoelectric point to result in a charged target analyte.
Damage to the nitrocellulose strip and the flow uniformity when integrating the external membranes for ICP. To avoid this, the charge and the way it is applied on the membrane may be tuned.

Multiplex LFA Strips and Programmable Actuation

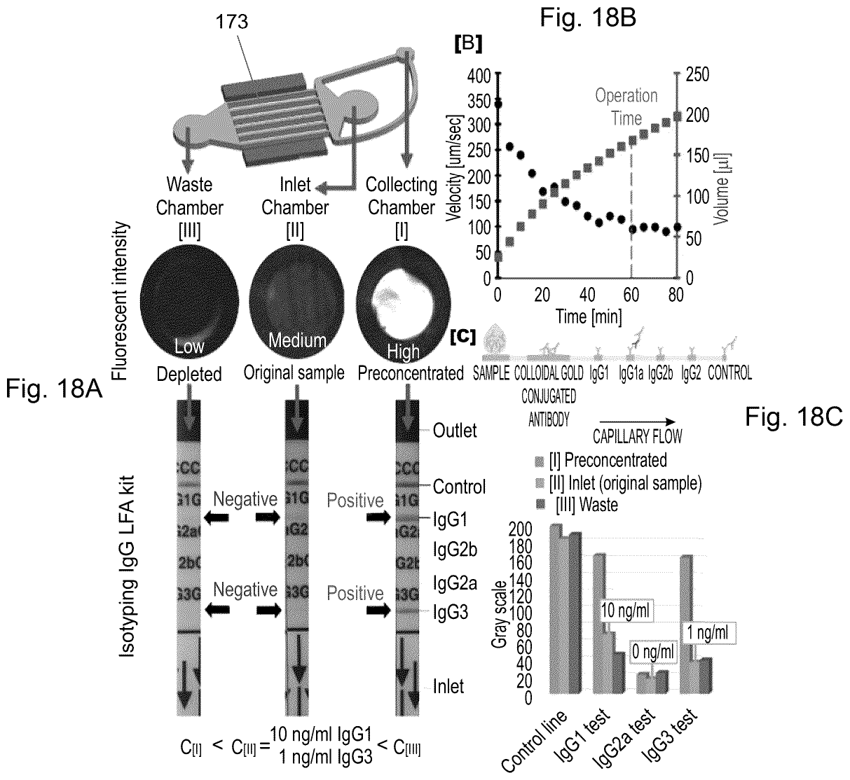

Most LFAs are designed to identify a single biomarker. Some commercial LFA kits provide for multiplex sensing by integrating several test lines, as shown in FIG. 18A. FIG. 18 A-C shows off-strip concentration. Detection of multiple analytes within a single sample enables healthcare providers to gain more information per test and to detect increasingly complex health conditions. Hence, multiplex LFAs are expected to become increasingly important in the future.

An on-strip platform with programmable ICP actuation for multiplex LFA: To enable enhanced-sensitivity on-strip multiplex, the present embodiments include an LFA strip holder that includes an array of individually addressable membrane pairs for each of the test lines (FIGS. 10A-B). The embodiment may provide for programmable control over the preconcentrated plug, as we demonstrate within fabricated microfluidic channels, see FIGS. 5A and 5B above.

Materials and Methods:
The housing and integration of the system is extended for multiple array membranes.
Target multiple analytes and multiplex LFA kit: Mouse isotyping IgG kit (IgG-1, IgG-2a, IgG-2b, IgG3).
Portable system: A portable housing box includes all electronic units, including a power supply (battery), printed circuit board (PCB), membranes, and a controller, for automatic operation of the multiple ICP actuations.

### Risks and Mitigation:

If nanocolloids cannot be preconcentrated, but only the biomolecules, we may tune the operation time and programmed actuation of the array so as to avoid full passage of the nanocolloids downstream to the waste pad before their interaction with the molecule plugs. This risk exists for a single test lines as well, but, in the case of multiple test lines, is more severe due to the potentially prolonged operation time.
Non-negligible EOF may be generated due to the electric fields that are generated in between multiple membrane pairs which can affect the capillary flow within the strip. The voltage and the number of actuated membranes may be ill be tuned so as to avoid the dominance of the EOF.

Off-Strip Preconcentration

The second embodiment for LFA devices is an off-strip strategy, which adds a preconcentration step before application of the sample to the LFA, and consists of an external microfluidic device that can be integrated into the LFA inlet, see FIG. 15B. As shown in FIG. 15B, a relatively large volume sample is added to concentration device 170. Within the device are one or more paths 172—see inset—and on each path there are individually addressable membranes that may be electrified to form charge plugs as above. The plugs cause a location to form of concentrate. Plug formation 173 may be aligned with an outlet which may be aligned with the inlet of the test device 174. This approach overcomes the restriction on the total sample volume that can be processed by a LFA test, thereby limiting the number of target molecules that pass through the strip and the resulting assay sensitivity, by processing a larger sample volume that undergoes volume reduction simultaneously to preconcentration of the target analyte. Additionally, in contrast to the on-strip ICP approach, it avoids the need to integrate the membranes directly in the LFA and to consider the buffer of the kit. Typically, to perform a test, commercial LFAs recommend introducing total solution volume of several hundreds of microliters to the inlet pad, of which only a few dozen microliters are of the biological sample that contains the target analyte, while the rest is the extraction/reaction buffer. Any deviation from this recommendation, e.g., increasing the biological sample volume, may result in operation under non-ideal conditions and eventually to reduced test quality. Thereby, the concentrator of the present embodiments may provide a strategy to process a higher biological sample volume without deviating from the optimized test conditions. An effective off-strip preconcentrator device may include optimization of the ICP device for high-throughput processing of the sample for reduced operation time as well as increasing the preconcentration factor, adjustments for various biological liquids with potential debris that can jam the device, and user-friendly integration of such a device into a commercially available LFA. Protection against jamming may involve using multiple paths, each using the same detectors.

Preliminary Results:

Using the device, we preconcentrated two analyte species, mouse IgG1 and fluorescent-tagged mouse IgG3, within 60 min (FIG. 18B). During this time a 1701 sample solution (200 μS/cm KCl, 10 ng/ml IgG1 and 1 ng/ml IgG3) was processed through the chip using hydrostatic pressure-driven flow, while the target analytes accumulated into a plug in a designated location (by applying 20V through the membranes to form ICP). This step was then followed by release of the preconcentrated plug into a collecting chamber in order to extract it from the microfluidic chip (a total volume of ˜5 μl) without it undergoing dilution. The extracted solution with the preconcentrated analytes was further mixed with the LFA (mouse isotyping IgG kit) buffer in accordance with the manufacturer's protocol, and introduced into the IgG-strip. Significantly enhance signals (˜10 times) at both IgG1 and IgG3 tests line were achieved (FIGS. 18B, C) when samples were first preconcentrated before applying them to the LFAs.

Optimization of the Off-Strip Microfluidic Pre-Concentrator:

The system's performance may be adjusted as needed for biological fluids and optimized using various designs and system parameters (e.g., voltage, membrane location, buffer). The experimental investigation may be complemented as in the above, with numerical simulations for better physical understanding.

It is noted that some biological fluids or analytes are unsuitable with ICP-based preconcentration (conductivity too high, low pH, uncharged analyte). In contrast to the on-strip approach, in the off-strip embodiment we are not restricted to work with the kit buffer and hence can modify buffer for our needs.

In the event of increase in false-positive readouts due to preconcentration of inhibitors that exist in biological fluids, hindering molecules may be identified and sorted or filtered out (e.g., electrokinetic sorting, immunoprecipitation etc.) as an additional step.

Target analyte preconcentration according to the present embodiments may be expected to significantly improve the sensitivity of commercial LFA devices to biomarkers at early stages of disease.

Currently, commercially available LFA kits do not integrate on- or off-strip electrokinetic preconcentration and the present embodiments may enable realization of such solutions. Previous studies incorporating electrokinetic preconcentration in paper-based devices have used customized cellulose papers that used reporters (e.g. fluorescent dyes) and configuration different than those commonly used in commercial LFA. Integration of an ICP-driven preconcentration mechanism into on-strip commercial LFAs may also enable the control over multiple plugs for multiplex sensing.

The proposed solution improve biomarker detection for early diagnosis of diseases.

The preconcentration technique according to the present embodiments may be applicable for any LFA paper strip device. It can be integrated in the form of a disposable off-strip preconcentrator or a holder that includes the electronics (battery, controller and optional optical detector) and membranes into which the disposable strip is inserted. The latter mode may also enable programmable control of the preconcentrated plug over several test lines for multiplex detection.

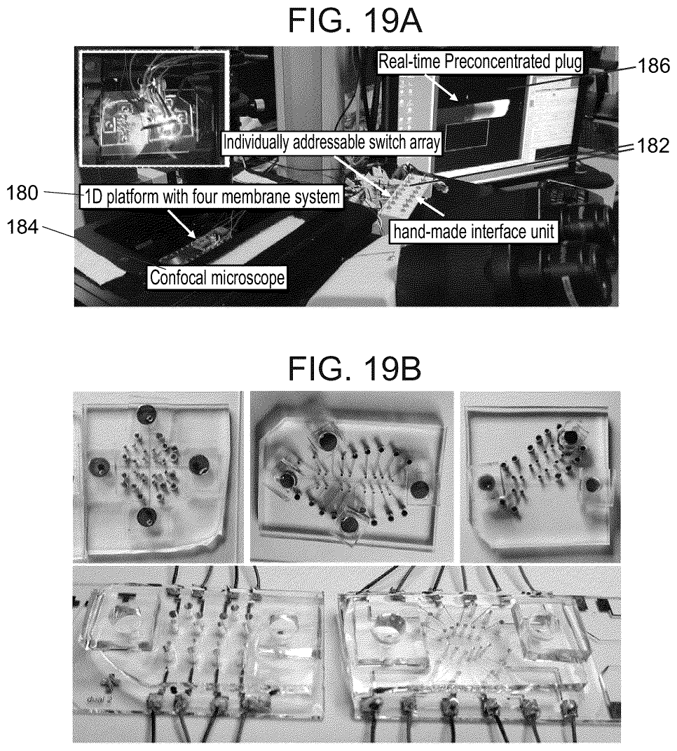

Reference is now made to FIGS. 19A and 19B, which shows experimental realizations of the microfluidic networks in FIGS. 1A-B, FIGS. 7A-E and FIGS. 8A-B above. A platform 180 with a membrane system is attached to an individually addressable switch array 182 which has electrical switches that address the individual membranes of the platform. Confocal microscope 184 determines the presence of the plug which is shown on screen 186. FIG. 19B shows realizations of the platform.

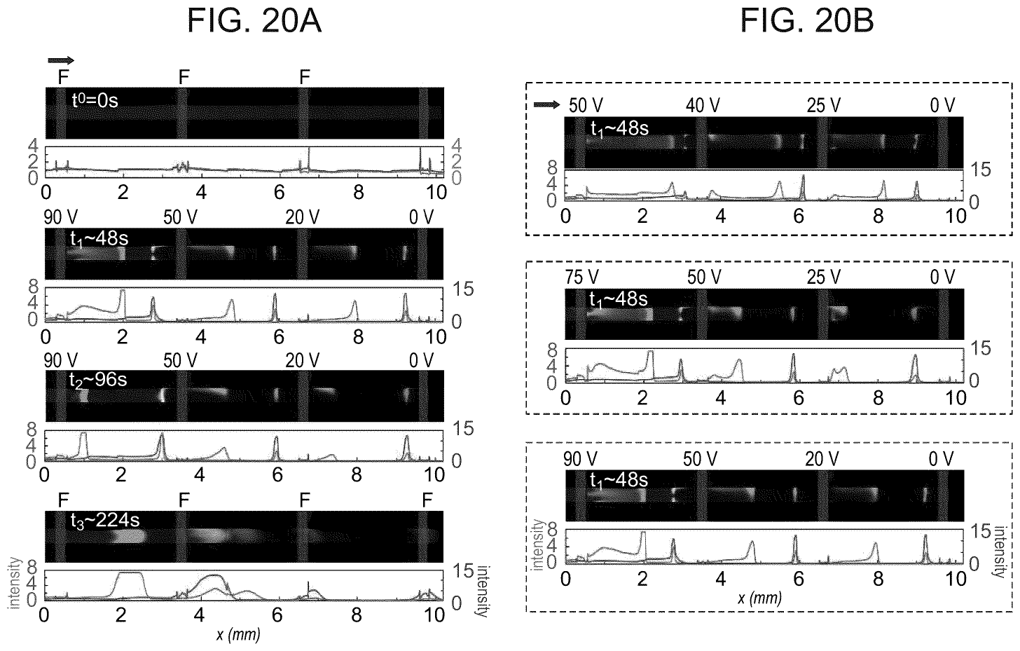

Reference is now made to FIGS. 20A and 20B. The figures schematically show separation of preconcentrated plugs of two species under different voltages applied between pairs of membranes within a 1D channel geometry. FIG. 20(A) shows superimposed fluorescence microscopy images of the separation of two fluorescent molecules (Dylight 488 and RFP) with different electrophoretic mobility manipulation, upon application of differential voltages on the four serial membranes (V₁, V₂, V₃, 0), and their corresponding green and red fluorescence intensity profiles. FIG. 20B is a comparison of separation behavior under various voltage difference conditions simultaneously applied to the membranes (t=48 s). The green and red lines in the graphs indicate the corresponding normalized (to their initial intensity value) fluorescence intensity profiles of Dylight 488 molecules and RFP, respectively.

It is expected that during the life of a patent maturing from this application many relevant test strips and preconcentration plugs will be developed and the scopes of these and other terms are intended to include all such new technologies a priori.

The terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment and the present description is to be construed as if such embodiments are explicitly set forth herein. Conversely, various features of the invention, which are, for brevity, described in the context of a single embodiment, may also be provided separately or in any suitable subcombination or may be suitable as a modification for any other described embodiment of the invention and the present description is to be construed as if such separate embodiments, subcombinations and modified embodiments are explicitly set forth herein. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. Device for concentration of bioparticles for identification comprising:
at least one capillary flow path from an inlet for advection of said bioparticles in a buffer solution;
at least one pair of membranes along said flow path, the membranes being individually selectable for electrical powering, thereby to controllably set up a region subject to a voltage gradient at a location along said path, said region causing localized concentration of said bioparticles into at least one preconcentrated bioparticles plug;
detection surface immobilized molecular probes located along said flow path to detect said bioparticles following said localized concentration.

2. The device of claim 1, wherein said device is a paper-based lateral flow device having at least one test line, and configured such that said localized concentration occurs at said test line.

3. The device of claim 1, wherein said device is a concentrator, configured with an outlet with said localized concentration, said outlet configured for extracting said localized concentration of bioparticles when said outlet is aligned to the inlet of a lateral flow device.

4. The device of claim 1, wherein said flow path comprises a microfluidic network of microfluidic channels.

5. The device of claim 1, wherein said at least two membranes comprise an array of membranes, each membrane being individually selectable for electrical powering.

6. The device of claim 5, configured such that changing a selection of powered membranes in said array maneuvers said localized concentration.

7. The device of claim 1, wherein said flow path comprises a one-dimensional path and said membranes are arrayed on said path.

8. The device of claim 1, wherein said flow path comprises a two-dimensional network and said membranes are arrayed over said two-dimensional network.

9. The device of claim 1, wherein said membranes comprise ion-permselective membranes.

10. The device of claim 1, wherein said flow path comprises a paper-based lateral flow assay.

11. The device of claim 6, configured such that said selecting of electrical powering on said membranes performs digital-like microfluidic operations on said at least one preconcentrated bioparticles plug.

12. The device of claim 11, wherein said digital-like microfluidic operations comprise one or more of: down and up-stream translations, splitting, merging, parallelization, and multiplex sensing of said at least one preconcentrated bioparticles plug.

13. The device of claim 12, configured to allow digital-like manipulations of multiple plugs containing different preconcentrated particles/molecules from samples introduced via separate inlets.

14. The device of claim 1, comprising individual electrodes to respective ones of said membranes, therethrough to selectively electrically power said membranes.

15. The device of claim 1, configured to differentially electrify said membranes to generate either an enrichment layer or a depletion layer.

16. The device of claim 15, configured such that, upon the application of a voltage drop between two of said membranes in the face of background net flow in said microchannel, a depletion layer is generated from the interface of the downstream membrane, thereby to cause said concentration localization to occur at an edge of said depletion layer.

17. The device of claim 1, comprising a serial array of at least three individually addressable membranes, and intermembrane spacings, embedded within a respective straight flow path.

18. A method of identifying bioparticles/biomolecules in a buffer fluid, comprising:
inserting the bioparticles/biomolecules and buffer fluid into a microfluidic network, the microfluidic network forming at least one capillary flow path;
differentially electrifying individually addressable membranes embedded into said flow path of said microfluidic network, to cause a concentration of said bioparticles/biomolecules into a first localized concentration plug; and
changing said electrifying of said membranes to hold or maneuver said concentration plugs around detection molecular probes.

19. The method of claim 18, further configured to carry out further changing of said electrifying to generate at least one additional concentrated plug.

20. The method of claim 19, comprising further changing said electrifying to carry out digital-like manipulation of said first and said at least one additional plug.
